(19) United States
(12) Patent Application Publication
(10) Pub. No.: US 2019/0046446 A1
(43) Pub. Date: Feb. 14, 2019

(54) APO-E MODIFIED LIPID NANOPARTICLES FOR DRUG DELIVERY TO TARGETED TISSUES AND THERAPEUTIC METHODS

(71) Applicant: Eriochem USA, LLC, Carson city, NV (US)

(72) Inventors: Jose Lucio NUNEZ, Parana (AR); Dante SELENSCIG, Parana (AR); Maria de los Angeles RAMIREZ, Parana (AR)

(73) Assignee: Eriochem USA, LLC, Carson City, NV (US)

(21) Appl. No.: 15/760,170

(22) PCT Filed: Sep. 28, 2017
(86) PCT No.: PCT/US17/54045

§ 371 Date: Mar. 14, 2018

Related U.S. Application Data
(60) Provisional application No. 62/402,632, filed on Sep. 30, 2016.

(51) Int. Cl. A61K 9/127; A61K 9/19; A61K 47/62; A61K 47/69; A61K 47/54; A61P 35/00; A61K 31/337

(52) U.S. Cl. CPC A61K 9/1275; A61K 9/19; A61K 9/1277; A61K 31/337; A61K 47/6929; A61K 47/548; A61P 35/00; A61K 47/62

(57) ABSTRACT

Lipid nanoparticles and methods of making and using the same, including pharmaceutical compositions and kits, and the targeted delivery of drugs in various treatment methods. The nanoparticle formulation includes phospholipids, triglycerides, cholesterol, cholesteryl ester, apolipoprotein E3 (ApoE3), and a lipophilic therapeutic agent.

### FIELD OF THE INVENTION

The invention relates to novel lipid nanoparticles with apolipoprotein for improved delivery of drugs to targeted tissues via LDL receptors. Also described are stable and lyophilized pharmaceutical compositions, a method to obtain the nanoparticles and a manufacturing procedure to obtain pharmaceutical compositions, kits comprising the nanoparticles, and therapeutic methods including administering effective amounts of the nanoparticles to patients in need thereof.

### BACKGROUND

Targeted therapies are treatments that target specifics cells, without harming other cells in the body. These therapies represent major improvements in the clinical treatment of many diseases, including cancer. Targeted therapies can lead to reduction of side effects (toxic effects) and reduction of dosage of administered drug, which results in less toxicity and costs.

Reports of targeting chemotherapeutics drugs using antibodies have appeared in the literature since 1958. Targeting drugs to antibodies for selective delivery to cancer cells has had a limited success due to the large size of the antibodies and their relative inability to penetrate the tumors cells; and alternative strategy comprises the use of smaller targeting ligands or peptides which recognize specific receptors.

Prior methods for delivering drugs generally include: (a) liposome-based methods, wherein the therapeutic agent is encapsulated within the carrier; (b) synthetic polymer-based methods for creating particles having precise size characteristics; and (c) direct conjugation of a carrier to a drug, wherein the therapeutic agent is covalently bound to a carrier (such as, e.g., insulin).

“Liposomes” are small particles that form spontaneously when phospholipids are sonicated in aqueous solution, and consist of a symmetrical lipid bilayer configured as a hollow sphere surrounding an aqueous environment. Liposomes have a large carrying capacity, but are generally too large to effectively cross the blood-brain barrier (BBB), for example. Furthermore, liposomes are inherently unstable, and their constituent lipids are gradually lost by absorption by lipid-binding proteins in the plasma. Accordingly, attempts have been made to direct liposomes to particular cellular targets. As an example, immunoliposomes have been constructed in a process that involves covalent attachment of monoclonal antibodies (mAbs) to the surface of the liposome.

Earlier studies have shown that the efficacy of liposome drug delivery was inversely related to the diameter of the liposome particle. That is, the average HDL particle has a diameter of 10-20 nm. Hence, even the smallest liposomes have a diameter five times larger than the average HDL particle.

Müller et al. (U.S. Pat. No. 6,288,040) describe the use of synthetic poly(butyl cyanoacrylate) particles to which ApoE molecules are covalently bound. The particle surface becomes further modified by surfactants or covalent attachment of hydrophilic polymers. Since these particles are not naturally occurring, they may have a variety of undesirable side effects. Furthermore, poly(butyl cyanoacyilate) is not an excipient approved by the FDA; and these particles use toxic surfactants such as Polysorbate 80 to cover the particle. Moreover, the described particles have a normal size of 300 nm. The presence of particles of about 300 nm of a synthetic material would likely trigger immune system responses.

Nelson et al. (U.S. Pat. No. 7,682,627) describes an artificial LDL for targeted carrier system for delivery across the blood-brain barrier, a method for manufacturing these particles and a method for producing conjugates of therapeutic agents with an LDL component to facilitate incorporation into LDL particle for transport across the BBB and subsequent release of the therapeutic agent into the cell. Conjugates include attachment of the therapeutic agent via an ester linkage that can be easily cleaved in the cytosol and consequently escape the harsh lysosomal conditions. These LDL particles comprised three elements: phospatidil choline, fatty-acyl-cholesterol esters, and at least one apolipoprotein.

McChesney et al. (U.S. Patent Application Publication No. 2015/0079189) describe synthetic LDL nanoparticles comprising mixtures of phospholipids, triglycerides, cholesterol esters, free cholesterol and natural antioxidants, for selective delivering of lipophilic drugs to cellular targets expressing LDL receptors after intravenous injection for cancer treatment. These synthetic low density lipoprotein nanoparticles are also described as a lipid emulsion with a shelf life at 25° C. greater than 1 year, or about 2 years when stored in a sealed container and away from the exposure of light.

These nanoparticles are prepared without any protein in order to avoid trigger clearance processes in the tissues of the reticuloendothelial system. Furthermore, these particles have a special coating layer that allows the particles to take the native lipoproteins as a coating; and after this coating the particles would be preferentially taken up by the targeted tissues.

There are teachings indicating that individuals have different levels of Apo proteins in the body, and these levels also could be affected by their physiological condition. Thus, the amount of ApoE available to be adsorbed in these nanoparticles would be different in each individual (Liu H. et al., 2015; Fidel Vila Rodriguez et al. (2011)). Thus, the proportion of nanoparticles that would take the Apo E from the bloodstream and eventually reach the targeted tissue will also depend on the physiological characteristics of each individual and their condition.

Going through the literature it can be found that the physicochemical characteristics of the nanoparticles and the compositions containing them are largely determined by the manufacturing method used to obtain them. In this sense, the manufacturing process for the particles described by Nelson et al. comprises different steps, such as: dissolving the lipids in methanol/chloroform (2:3); sonicating the solution for 1 hour that generates material contamination with titanium (see BETTS et al., Environmental Toxicology and Chemistry, Vol. 32, No. 4, pp. 889-893), a centrifugation in a potassium bromide (KBr) step gradient making it not pharmaceutically acceptable. Furthermore, some of the steps involved in the process are not scalable; e.g., the centrifugation step requires 285,000 g for 18 h; and the final step of dialysis against PBS to remove the KBr. Also, some of the manufacturing steps described by Nelson et al. are carried at a temperature over 50° C. which can lead to oxidation of the lipid components, and increased impurities of active ingredients used above values permitted for use.

In addition, Nelson et al. describes nanoparticles that can be stored at 4° C. preferably up to two weeks. In contrast, there is a need for a nanoparticle composition that is stable up to two weeks stored at 4° C. and for at least 18 months after lyophilization.

Many existing chemotherapeutic drugs, repurposed drugs and newly developed small molecule anticancer compounds which have high lipophilicity and low water-solubility are generally solubilized using high concentrations of surfactants and co-solvents, which frequently lead to adverse side effects. Nanoemulsions are kinetically stable and suitable for parenteral delivery of poorly water-soluble anticancer drugs. In comparison to other nanocarriers, nanoemulsions are easier to prepare and do not necessarily require organic solvent/co-solvents; so the risk of carrier toxicity is low. However, nanoemulsions are manufactured using high energy procedures, such as sonication or high pressure homogenization and the nanoformulations often include multiple components to achieve several functions. Their scale-up production thus becomes significantly more costly and technically difficult since most commonly used laboratory techniques (such as sonication) are difficult to implement at production scale. It also quite challenging to achieve nanoparticles with the same size in a larger batch. (See Narvekar M. et al., AAPS Pharm. SciTech., Vol. 15, pp, 4822-833 (2014)).

What is needed in the art is a product and manufacturing process which can be both scalable and pharmaceutically accepted and allows obtaining chemically and physically stable formulations comprising the nanoparticles.

Docetaxel (commercially marketed as TAXOTERE) is a well-known chemotherapeutic antimitotic clinical drug that works by preventing cell multiplication. It has been approved for the treatment of locally advanced or metastatic breast cancer, head and neck cancer, gastric cancer, hormone refractory prostate cancer and non-small cell lung cancer. It can be used in combination with other chemotherapeutic drugs, depending on the specific type of cancer and its stage of severity.

Unfortunately, TAXOTERE has an unpredictably high interindividual variability, both in efficacy and in toxicity, which has been associated with its pharmacokinetic variability. It also has resulted in reactions of unpredictable acute toxicity in an incidence range of 5-60% with severity of manifestation ranging from medium itching to systemic anaphylaxis. Additionally, it has been found to cause fluid retention with weight gain, peripheral edema and occasional pleural or pericardial effusions, which has been reported at an incidence rate of 50% or higher for cumulative doses of docetaxel of 400 mg/m² or greater (See J. Clin. Oncol. 14: 422-8, 1996; J. Clin. Oncol. 16: 187-96, 1998; J. Natl. Cancer Inst. 87: 676-81, 1995).

The occurrence of TAXOTERE hypersensitivity reactions has been attributed, at least in part, to Polysorbate 80 (Agents Actions 12: 64-80, 1982; Contact Dermatitis 37:0-18 (1997)). Fluid retention is related to the fact that Polysorbate 80, which increases membrane permeability (Eur. J. Biochem., 228: 1020-9, (1995)), also increases plasma viscosity and erythrocyte morphology, thus contributing to their cardiovascular side effects (Br. J. Pharmacol., 134: 1207-14, 2001). Furthermore, TAXOTERE is a product made from vegetable raw materials that do not allow for easy removal of impurities, and this may be a possible cause of the fluid retention, which also decreases the therapeutic index of the drug.

Therefore, what is also needed is a Polysorbate 80-free Docetaxel formulation that does not have the manifested toxicity of TAXOTERE and ideally has improved biological activity.

### SUMMARY OF THE INVENTION

It is an object of this invention to overcome the challenges encountered during delivery of certain therapeutic agents (drugs). Accordingly, described herein are lipid nanoparticles comprising ApoE3, which are suitable for delivering one or more therapeutic agents for treatment of cancer. Also the invention describes stable lyophilized pharmaceutical compositions and kits comprising the nanoparticles. In still additional embodiments, the invention relates to a manufacturing process for producing the nanoparticle, as well as associated therapeutic methods for using the nanoparticles and pharmaceutical compositions comprising the same.

It is furthermore an object of the invention to develop a new Polysorbate 80-free Docetaxel formulation that avoids the manifested toxicity of the original formulation of Docetaxel and presents improved behavior for treatment, particularly for treatment of cancers associated with over-expression of r-LDL receptors, such as lung and prostate cancer.

### BRIEF DESCRIPTION OF THE DRAWINGS

FIG. 1 is an illustration of a configuration of the lipid nanoparticle according to an exemplary embodiment of the invention.

FIG. 2 is a flow diagram of a representative manufacturing method of the lipid nanoparticles according to embodiments of the invention.

FIG. 3 illustrates representative manufacturing equipment for manufacture of the lipid nanoparticles according to embodiments of the invention.

FIG. 4A-4D shows the volume distribution of nanoparticles loaded with Docetaxel according to exemplified embodiments of the invention

FIGS. 5A-5C show stability results in terms of Z-average, PDI and Docetaxel content after 6, 12, and 18 months of the lipid nanoparticles according to embodiments of the invention.

FIG. 6 shows in vitro release over time according to exemplified embodiments of the invention for Docetaxel (TAXOTERE), and for Nanoparticle loaded with DCX with and without ApoE3.

FIG. 7 shows the tolerability of lipid nanoparticles with and without ApoE3, both containing no Docetaxel, in a single-dose tolerability study in healthy New Zealand rabbits based on serum biochemistry parameters for gamma-glutamyltransferase (GGT) in FIG. 7A and for glutamic oxaloacetictransaminase (GOT) in FIG. 7B.

FIG. 8 shows GGT (FIG. 8A) and GOT (FIG. 8B) concentrations in plasma 24 hours after inoculation with (A) DCX, (B) Nano+DCX+ApoE3, (F) Nano+DCX, and (H) PBS.

FIGS. 9A-9D show size distribution for nanoparticles manufactured with different types and amounts of triglycerides.

FIG. 10 shows the size distribution by volume of lipid nanoparticles according to embodiments of the invention.

FIGS. 11A-11G show immunogenicity results in terms of optical density by Logarithm of the serum concentration of antibodies anti-ApoE3 according to embodiments of the invention.

FIG. 12 shows PotentialZ (mV) changes by the concentration of ApoE3 Present in the lipid nanoparticle according to embodiments of the invention.

FIG. 13 shows nanoparticle size distribution changes in terms of volume by Size for changes in the ApoE3 concentration in the lipid nanoparticle according to embodiments of the invention.

FIGS. 14A-14C are graphs showing absorbance vs. Docetaxel concentrations for (A) PC-3 cells, (B) A549 cells, and (C) VERO cells.

FIGS. 15A-15C are graphs showing absorbance vs. Docetaxel concentrations for (A) PC-3 cells, (B) A549 cells, and (C) VERO cells as in FIGS. 14A-C, except replacing normal fetal bovine serum was replaced with lipoprotein-free serum.

FIGS. 16A-16B are graphs showing Docetaxel concentrations in plasma samples at different times after administration of (A) TAXOTERE, or (B) Nano+DCX+ApoE3. FIG. 16C shows concentration of Docetaxel 24 hours after intravenous administration of TAXOTERE (T) or Nano+DCX+ApoE3 (NDA).

### DETAILED DESCRIPTION OF EMBODIMENTS OF THE INVENTION

The present invention may be understood more readily by reference to the following detailed description of the preferred embodiments of the invention. However, although different components and methods are disclosed and described, it is to be understood that this invention is not limited to specific formulations, assemblies or configurations, conditions, or methods, as such may vary, and any modifications thereto and variations therein will be apparent to those skilled in the art. It is also to be understood that the terminology used herein is for the purpose of describing specific embodiments only and is not intended to be limiting.

### I. Definitions

It must be noted that as used herein and in the appended claims, the singular forms “a,” “an,” and “the,” include plural forms unless the context clearly indicates otherwise. Thus, for example, reference to “a material” includes one or more of such same or different materials, and reference to “the method” includes reference to equivalent steps and methods known to those of ordinary skill in the art that could be modified or substituted for the methods described herein.

As used herein, the term “lipid binding protein” means a protein which may be associated with the phospholipids monolayer of the nanoparticle, preferably an apolipoprotein, including (but not limited to) ApoA, ApoB, ApoC, ApoD, ApoE, and all isoforms of each. As used herein, the term “ApoE” means one or more of the isoforms of ApoE, including but not limited to ApoE2, ApoE3, and ApoE4. In certain embodiments of the invention, ApoE3 is used as the apolipoprotein of the lipid nanoparticles.

“Controlled release” as used herein refers to release of a drug (therapeutic agent) from the nanoparticle so that the blood or tissue levels of the pharmaceutically active ingredient is maintained within the desired therapeutic range for an extended period (hours or days).

As used herein, “Docetaxel” refers to the chemotherapeutic antimitotic clinical drug, which is commercially marketed under different names. When used within specific Examples herein, Docetaxel specifically refers to the TAXOTERE formulation used.

“Nanoparticles” are particles with a diameter of less than about 1,000 nm (1 μm) comprising of various biodegradable or non-biodegradable polymers, lipids, phospholipids or metals. (See Jin, Y., Nanotechnology in Pharmaceutical Manufacturing, Pharmaceutical Manufacturing Handbook: Production and Processes. Vol. 5., Section 7, John Wiley & Sons, 200; and Lockman, P. R., et al., “Nanoparticle technology for drug delivery across the blood-brain barrier.” Drug Development and Industrial Pharmacy 28.1: 1-13 (2002)).

“Nanoemulsion” as used herein refers to a nanosized colloidal systems that consists of poorly water soluble compounds, suspended in an appropriate dispersion medium (oil-in-water emulsion) stabilized by surfactants.

As used herein, the terms “therapeutic agent” and “active ingredient” means therapeutically useful amino acids, peptides, proteins, nucleic acids, including but not limited to polynucleotides, oligonucleotides, genes and the like, carbohydrates and lipids. The therapeutic agents according to embodiments of the invention may include neurotrophic factors, growth factors, enzymes, antibodies, neurotransmitters, neuromodulators, antibiotics, antiviral agents, antifungal agents and chemotherapeutic agents, and the like. The therapeutic agents of the present invention include drugs, prodrugs, diagnosis substances, contrast agents and precursors that can be activated when the therapeutic agent is delivered to the target tissue.

As used herein, the term “pharmaceutically acceptable carrier” means a chemical composition or compound with which an active ingredient may be combined and which, following the combination, can be used to administer the active ingredient to a patient. In embodiments, “pharmaceutically acceptable carrier” also includes, but is not limited to, one or more of the following: excipients, surface active agents, dispersing agents, inert diluents, granulating and disintegrating agents, binding agents, lubricating agents, sweetening agents, flavoring agents, coloring agents, preservatives, physiologically degradable compositions such as gelatin, aqueous vehicles and solvents, oily vehicles and solvents, suspending agents, dispersing or wetting agents, emulsifying agents, demulcents, buffers, salts, thickening agents, fillers, antioxidants, stabilizing agents, and pharmaceutically acceptable polymeric or hydrophobic materials.

As used herein, “an effective amount” refers to the amount sufficient to bring about a desired result in an experimental setting. A “therapeutically effective amount” or “therapeutic dose” refers to an amount sufficient to produce a therapeutic response or beneficial clinical result in a patient.

As used herein, the terms “patient” and “individual” refer to any person or other subject that is need of, and would receive a benefit from, administration of the lipid nanoparticles according to therapeutic methods described herein. It is envisioned that the “patient” may also be a non-human animal, such as, e.g., in veterinary applications of the invention.

As used herein, the term “therapeutic index” or “TI” refers to a comparison or the ratio of the amount of a therapeutic agent that causes the therapeutic effect to the amount that causes toxicity, and is calculated as TI=LD50/ED50 (lethal dose 50/effective dose 50).

As used herein, the term “Selectivity Index” refers to a comparison or ratio between the IC50 in non-cancer cells and the IC50 in cancer cells. This IS value shows the differential activity of a product between healthy and non-healthy cells. The higher the value, the more selective the product will be.

### II. Lipid Nanoparticles

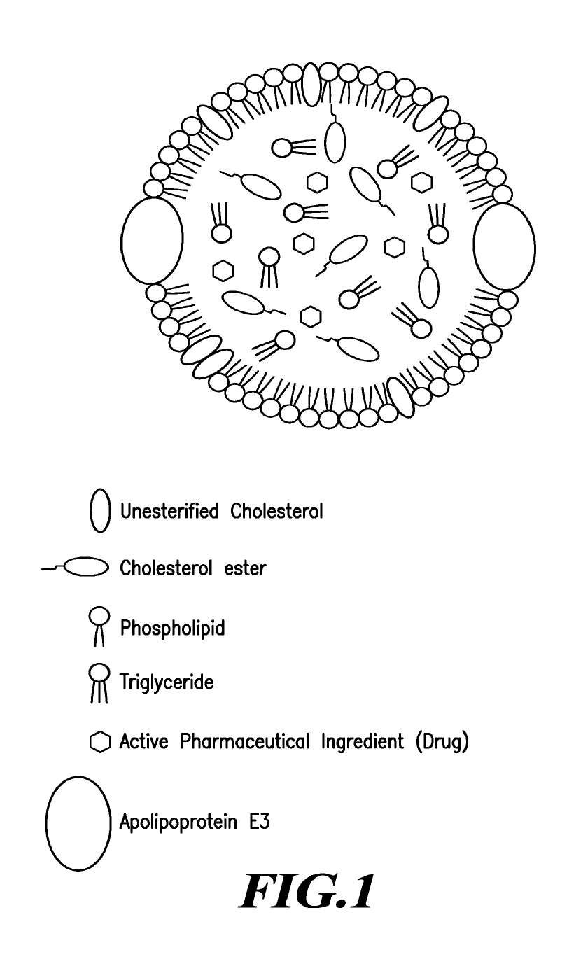

The structure/configuration of a lipid nanoparticle of the invention is depicted in FIG. 1. The ingredients are distributed so as to form a lipid core, covered by a phospholipid layer, and finally a surfactant coating layer. The active pharmaceutical ingredient, or a lipophilic active ingredient, is located in the lipid core or the phospholipid layer; and a lipid binding protein (e.g., ApoE3) is bonded to the surface of the nanoparticle.

In embodiments, the lipid core of the nanoparticle is non-aqueous and has a high retention capacity for the lipophilic (or liposoluble) active ingredient(s). The lipid binding protein is preferably an apolipoprotein, such as ApoE3 or analogs thereof. In preferred embodiments, the apolipoprotein is recombinant ApoE3 and may be further modified to enhance targeting efficacy of the active ingredient(s).

The lipid nanoparticles may be spherical, oval, or discoid in shape and have a diameter of about 20-150 nm, such as 30-80 nm.

In one aspect, the invention relates to the specific composition of ingredients that results in the stable nanoparticle having the structural characteristics desirable for drug delivery. That is, the structure and behavior of the nanoparticle are consequences of their composition.

Lipids suitable for use in nanoparticles of the invention include (but are not limited to) phospholipids, triacylglycerols, cholesterol, cholesterol esters, fatty-acyl esters, and the like. Preferably, nanoparticles of the invention are generally formed of the following five components: (1) phospholipid, (2) triglyceride, (3) cholesterol ester, (4) cholesterol, and (5) ApoE3. For example, in a preferred embodiment, the lipid core may be made of cholesterol ester and triglyceride (e.g., castor oil), the phospholipid layer may be made of egg yolk phospholipid, and the surfactant coating layer may be made of sodium taurodeoxicholate and Poloxamer188.

In certain embodiments the nanoparticles of the present invention are loaded with Docetaxel in combination with human recombinant ApoE3. As compared to Docetaxel in its original formulation with Polysorbate 80, the lipid nanoparticles of the invention have lower IC50 and a higher selectivity index in human lung cancer and human prostate cancer cell lines in lipoprotein free serum, thus providing a novel and improved treatment option for these cancers, as discussed further below.

A. Phospholipids

Phospholipids suitable for use in the nanoparticles include (but are not limited to) diacylgliceride structures and phosphophingolipids. Diacylglycerides structures include phosphatidicacid (phosphatidate) (PA); phosphatidylethanolamine (cephalin) (PE), phosphatidylcholine(lecithin) (PC), phosphatidilserine (PS) and phosphoinitides. The Phosphosphingolipids include Ceramide phosphorylcholine (Sphingomyelin) (SPH), Ceramidephosphorylethanolamine (Sphingomyelin) (Cer-PE) and Ceramide phosphoryl lipid. The phospholipids suitable for use in the nanoparticles formulation include natural phospholipid derivatives and synthetic phospholipid derivatives. Natural phospholipid derivates include egg PC, egg PG, soy PC, hydrogenated soy PC and sphingomyelin. Synthetic phospholipid derivatives include: Phosphatidic acid; Phosphatidylcholine; 1,2-Didecanoyl-sn-glycero-3-phosphocholine (DDPC); 1,2-Dilauroyl-sn-glycero-3-phosphocholine (DLPC); 1,2-Dimyristoyi-sn-glycero-3-phosphocholine (DMPC); 1,2-Dipalmitoyl-sn-glycero-3-phosphocholine (DPPC) 1,2-Distearoyl-sn-glycero-3-phosphocholine (DSPC); 1,2-Dioleoyl-sn-glycero-3-phosphocholine (DSPC); 1-Palmitoyl-2-oleoyl-sn-glycero-3-phosphocholine (POPC); 1,2-Dierucoyl-sn-glycero-3-phosphocholine (DEPC), Phosphatidylglycerol (DMPG); 1,2-Dimyristoyl-sn-glycero-3-phosphoglycerol; 1,2-Dipalmitoyl-sn-glycero-3-phosphoglycerol (DPPG); 1,2-Distearoyl-sn-glycero-3-phosphoglycerol (DSPG); 1-palmitoyl-2-oleoyl-sn-glycero-3-phosphoglycerol (POPG); Phosphatidylethanolamine (DMPE); 1,2-Dimyristoyl sn-glycero-3-phosphoethanolamine; 1,2-Dipalmitoyl-sn-glycero-3-phosphoethanolamine (DPPE), 1,2-Distearoyl-sn-glycero-3-phosphoethanolamine (DSPE); 1,2-Dioleoyl-sn-glycero-3-phosphoethanolamine (DOPE).

In an embodiment, phospholipids suitable for use in the nanoparticles comprise 1,2-Dimyristoyl-fin-glycero-3-phosphocholine (DMPC), Phosphatidylglycerol (DMPG); 1,2-Distearoyl sn-glycero-3-phosphocholine (DSPC); 1,2-Distearoyl-sn-glycero-3-phosphoglycerol (DSPG); and egg PC. In one embodiment, the phospholipid is egg PC.

B. Triglycerides

Triglycerides suitable for use in the nanoparticles formulation include (but are not limited to) triglycerides which are liquid at room temperature. Triglycerides suitable for use in the nanoparticles are selected from the group comprising canola oil, castor oil, chia seed oil, coconut oil, corn oil, cottonseed oil, olive oil, palm oil, peanut oil, safflower oil, sesame oil, soybean oil and others. Triglycerides also include Mono-, di- and tri-acyl glycerols, were the fatty acids can be Mono-unsaturated fatty acid (Palmitoleic acid, Oleic acid, Elaidic acid, Gadoleic acid, Eicosenoic acid, Erucic acid and others), Di-unsaturated fatty acid (Linoleic acid, Eicosadienoic acid, Docosadienoic acid and others) and Polyunsaturated fatty acids (Linolenic acid, Dihomo-γ-linolenic acid, Eicosatrienoic acid, Stearidonic acid, Arachidonic acid, Eicosatetraenoic acid, Eicosapentaenoic acid, Tetracosanolpentaenoic acid, Docosahexaenoic acid and others). The di- and tri-acyl glycerols can contain or not identical fatty acids. Fractionated triglycerides, modified triglycerides, synthetic triglycerides, hydrogenated triglycerides and mixtures of triglycerides are also within the scope of the invention and mixtures thereof.

In some embodiments, triglycerides suitable for use in the nanoparticles comprise castor oil, soy oil, coconut oil, and/or hydrogenated castor oil. In certain embodiments, the triglyceride of the nanoparticles is castor oil, and the therapeutic agent is dissolved in this component within the nanoparticle core.

C. Cholesterol and Cholesterol Esters

Cholesterol esters refer to cholesterol esterified with saturated fatty acid, including (but not limited to) myristic acid, palmitic acid, stearic acid, arachidic acid, lignoceric acid, and the like, or an unsaturated fatty acid, including but not limited to palmitoleic acid, oleic acid, vaccinic acid, linoleicacid, linolenic acid, arachidonic acid, eicosatrienoic acid, stearidonic acid, arachidonic acid, eicosatetraenoic acid, eicosapentaenoic acid, tetracosanolpentaenoic acid, docosahexaenoic acid and the like.

In some embodiments, the cholesterol ester of the nanoparticles is cholesteryl oleate. The cholesterol esters are located in the lipid core, whereas cholesterol is located in the phospholipid layer. Cholesterol is used in a proportion of between 0 and 4° % of the nanoparticle components.

D. Lipid Binding Protein: Apolipoprotein

In compositions of the invention, the surface of the nanoparticles has bonded the lipid binding protein, preferably an apolipoprotein such as ApoE3. The apoprotein molecule is responsible for binding to lipoprotein receptors in the targeted tissues. According to Mims et al. depending on the state of the lipid constituents, the apoproteins undergo structural changes. (Minis et al., Biochemistry 29(28): 6639-47 (1990)).

ApoE is an apoprotein involved in cholesterol transport and plasma lipoprotein metabolism throughout the body. In peripheral cells, ApoE influences cellular concentrations of cholesterol by directing its transport. In neurons, changes in cholesterol levels influence the phosphorylation status of the microtubule-associated protein at the same sites that are altered in Alzeheimer's disease. This apoprotein has three major isoforms: ApoE4, ApoE3, and ApoE2, differing by single amino acid substitutions. At physiological concentrations (micromolar), ApoE exists predominantly as a tetramer. In a lipid-free state, the carboxy-terminal domain of the apolipoprotein forms a dimer, which then dimerizes to form the tetramer. However, ApoE is likely to bind to lipids in its monomeric, rather than tetrameric, state. (See Hatters et al., Apolipoprotein E Structure: Insights Into Function, Journal of Biological Sciences, 31(8), 445-454 (2006); and Peters-Libeu et al., Model of Biologically Active Apolipoprotein E Bound to Dipalmitoylphosphatidylcholine, Journal of biological Chemistry 281(2), 1073-79 (2006)).

In preferred embodiments, recombinant ApoE3 is used as the apolipoprotein component. Preferably, the nanoparticles comprise recombinant or cloned ApoE3 which may be further modified to enhance targeting efficacy. The use of recombinant ApoE3 avoids problems with antigenicity due to possible post-translationally modified, variant, or impure ApoE3 protein purified from human donors.

McChesney et al. described synthetic LDL prepared with any protein wherein the nanoparticle becomes coated with native apolipoprotein upon intravenous injection and is recognized and internalized by cellular LDL receptors. In this regard, and as as previously stated, there is information showing that each individual has different levels of Apo proteins in the body, and these levels also vary depending on the physiological conditions. (See Liu et al., 2015; Vila-Rodriguez et al., 2001; Robitaille et al., 1996; Valdez et al., 1995; Haffner et al., 1996; Utermann et al., 1987). Thus, depending on the amount of Apo proteins available and the predominant isoform in each individual, this can result in a large variability of the results, which is not desirable for a pharmaceutical composition and therapeutic uses.

In embodiments of the invention, the recombinant ApoE3 has a high affinity for the exposed surface of the nanoparticles and therefore sticks to the nanoparticles under the specific conditions discussed in connection with the manufacturing method. The average size of the ApoE3 is about 10.67±2.02 nm (n=4, media±SEM), and a Z-potential of the ApoE3 is about −14.47±1.18 mV (n=4, media±SEM), at pH 7.4.

Although the specific lipid components stated above may be preferred, embodiments of the invention may include other lipids, for example to include chemically-modified lipids, or admixtures of other naturally occurring lipophilic molecules that may work equally well. Persons skilled in the art will understand that modifications may be made to adapt the nanoparticles for a specific therapeutic agent or therapeutic application.

In the inventive nanoparticles, the ApoE3 may be present in an amount as low as 1% or less and does not require Polysorbate 80 for adhesion to the surface. In preferred embodiments, the nanoparticles do not contain any Polysorbate 80.

E. Therapeutic Agent

The nanoparticles may include one or more hydrophobic therapeutic agents. Specifically, it is an object of the invention to provide for natural and safe delivery of drugs that are highly toxic for human tissues, such as cancer treatment drugs. In embodiments, the therapeutic agent is a lipophilic drug and preferably an anticancer drug, and is preferably dissolved in the lipid core of the nanoparticles.

The therapeutic agent may be an anticancer agent selected from the group consisting of taxane, abeo-taxane, and other molecules derived from taxanes. In certain embodiments, the anticancer agent may include, e.g., paclitaxel, docetaxel, cabazitaxel, and the like.

In preferred embodiments, the therapeutic agent is an anti-cancer agent, or chemotherapeutic drug. Specifically, the therapeutic agent may be an anti-cancer or chemotherapeutic drug, suitable for treatment of metastatic breast cancer, head and neck cancer, gastric cancer, prostate cancer and lung cancer. In still further embodiments, the therapeutic agent is the chemotherapeutic antimitotic drug, Docetaxel, for treatment of lung and/or prostate cancer, particularly because these cancer tissues usually over-express r-LDL.

With respect to the above embodiment (Docetaxel), LDL receptor mediated uptake by certain cancer tumors/tissues plays in important role in the novel therapeutic uses and utility of the present invention. Specifically, the binding of Docetaxel to human plasma proteins was studied by ultrafiltration at 37° C. and pH 7.4 where Docetaxel was highly bound (>98%) to plasma proteins. At clinically relevant concentrations (1-5 μg/mL), the plasma protein binding rate was independent of the concentration. Due to lipoproteins alpha-1 acid glycoprotein and albumin being the main plasma Docetaxel transporters, and due to the high interindividual variability in the plasma concentration of the alpha 1-acid glycoprotein plasma, it was concluded that the alpha-1 acid glycoprotein should be the main determinant of the plasma variability of Docetaxel. (See S. Urine et al., Docetaxel Serum Protein Binding With high Affinity to Alpha 1-Acid Glycoprotein, Invest New Drugs, 2:147-51 (1996)).

Furthermore, elevated LDL receptor mediated uptake by certain tissue culture cells and experimental tumors from human lung cancer has been demonstrated in in vivo animals. In one study, ten patients with newly diagnosed lung tumors scheduled for surgery received an i.v. injection of LDL labeled with [14C] sucrose. After cell uptake and degradation of the LDL particle, the remaining radiolabeled sucrose was found to remain trapped in the lysosomal compartment, making this labeling technique useful for in vivo studies of LDL tissue absorption. Radioactivity was determined in plasma and in tissue biopsies obtained at surgery 1-3 days after injection. In 7 of 9 patients with primary lung cancer, absorption of radioactivity in lung cancer tissue rose 1.5-3.0 times compared to surrounding tissue. (See Sigurd Vitols et al, Elevated Uptake of Low Density Lipoproteins by Human Lung Cancer Tissue In Vivo, Cancer Research 52: 6244-47, November 15, (1992)). Furthermore, Urien et al. observed that free Docetaxel (fu) is 0.082 μM (0.066 μg/ml) at 1.24 μM (1 μg/ml) plasma serum—its characteristic in vivo therapeutic concentration. (Urien et al., Invest New Drugs, 1996).

Pursuant to the foregoing, an object of the invention is to provide a novel product of Polysorbate 80-free Docetaxel to avoid its manifested toxicity and with an improved selectivity index, with transport directed via r-LDL-mediated endocytosis because lung and prostate cancer tissues usually over express r-LDL. Therefore, in preferred embodiments is provided a formulation of lipid nanoparticles as described herein, having a mass ratio of about 1.2-2, such as 1.3-1.7, about 1.5 or preferably 1.4 of Docetaxel (MW 808)/ApoE3 (MW 34000), with a molar ratio of 40-80, or preferably 60 of Docetaxel molecules per each recombinant ApoE3 molecule.

In Experiments discussed further below, pharmacokinetics in rabbits comparing Docetaxel (TAXOTERE) and nanoparticles of the invention loaded with Docetaxel (DCX) and ApoE3 (Nano+DCX+ApoE) show that the inventive Nano+DCX+ApoE formulation has a greater clearance than TAXOTERE, likely due to TAXOTERE being strongly bound to plasma proteins whereas Nano+DCX+ApoE is more easily distributed in the target tissues. Furthermore, the Nano+DCX+ApoE according to embodiments of the invention has an absorption rate similar to TAXOTERE, but its absorption is relatively incomplete and with a rapid and fleeting response rate. As also shown by Examples below, pharmacodynamics in rabbit liver comparing TAXOTERE and Nano+DCX+ApoE show that at 24 hours, the latter has a higher concentration compared with TAXOTERE, while this concentration is similar at 36 hours.

In lung and prostate cancer cell cultures specifically, TAXOTERE has an IC50 of 34 and 30 μM, respectively, such that the presence of lipoproteins makes it 3.8 and 7.5 times more toxic, respectively. This further suggests that the cytostatic action of the TAXOTERE formulation would be influenced by the variable degree of hypocholesterolemia associated with these diseases, and the low concentration of Docetaxel that actually dissolved in plasma (i.e., free Docetaxel) would not seem to be responsible for its cytostatic action. That is, the activity of Docetaxel (TAXOTERE) in the nanoparticle formulation according to embodiments of the invention is much less influenced by the concentration of lipoproteins (IC50 of 16 and 21 μM vs 19 and 7 μM; and 0.84 and 3 times more toxic).

The amount of therapeutic agent present in the nanoparticles will vary in different embodiments of the invention, particularly depending on the therapeutic agent used. However, for optimal incorporation into the nanoparticle, the amount of therapeutic agent should be 1 gram drug per 20-40 grams of lipids (total lipid content); or 1 gram drug per 10-25 grams of Triglycerides; or 1 gram of drug per 7-15 grams of phospholipids. Multiple therapeutic agents or additional agents may be present in the core of the same particle, depending on the desired therapeutic objective.

The therapeutic agent, or lipophilic active ingredient(s), are encapsulated by the nanoparticles, and preferably dissolved in the triglyceride component. Notably, no covalent modification of the therapeutic agent is required for incorporation in the nanoparticles. In preferred embodiments, the therapeutic agent is not conjugated with another molecule within the core. That is, the lipid core of the nanoparticles has high retention capacity for liposoluble active ingredients without the need for conjugation. This is yet another advantage of the nanoparticle and the manufacturing process thereof according to embodiments of the invention, as there is evidence showing differences in activity between conjugated and non-conjugated therapeutic agents. For instance, there is evidence suggesting decreased activity of some drugs when the therapeutic agent is conjugated. There are results that for paclitaxel bonded to oleic acid the IC50 increases 10 compared with free drug. These mean that it takes 10 times more conjugated drug to produce the same effect than the free drug. (See Feng, Lan et al., 2011; Lundberg B. et al., 2003; Rodrigues, G., et al., 2005). Moreover, conjugation of a therapeutic agent requires a chemical reaction, or at least one additional step during the manufacturing process, which is obviously not needed for the preparation of the nanoparticles described herein.

Nelson et al. describe nanoparticles where the phospholipids and lipids are added in a ratio of between 11.5:1 and 12.5:1; and obtaining nanoparticles with a diameter of between 10 and 50 nm. As shown in Table 1, the charge capacity of these synthetic LDL is only 10% greater than the particles according to an exemplified embodiment of the invention. Furthermore, Nelson achieves that loading capacity by conjugating the active ingredient with cholesterol; while no covalent bond is needed for loading the inventive nanoparticles.

TABLE 1

| | Nelson et al. | McChesney | Inventive Nanoparticle |
|---|---|---|---|
| Phospholipids | 84% | 78% | 37% |
| Triglycerides | — | 10 | 56% |
| Cholesterol Esters | — | 2 | 1% |
| Cholesterol | 4% | 1 | 2% |
| Apo E | 8% | — | 1% |
| Active Ingredient/ | 4% | 9% | 3% |
| Therapeutic Agent | | | |
| Particles Size | 10-50 nm | 40-80 nm | 20-150 nm |

F. Concentration Ratios

In an embodiment, the lipid nanoparticles of the invention comprise a mixture of the components enumerated above. It has been found that the presence of the five ingredients described above, in specific concentrations, results in the inventive nanoparticles having the desirable characteristics described further herein. That is, as additionally demonstrated in the various Examples below, the specific concentration ratios of the respective components, as well as the presence of ApoE3, are critical to achieving the advantageous results that are unexpected over conventional nanoparticle formulations.

Specifically, the concentration ranges for the respective components, and the resulting ratios thereof, have been found to have an unexpected and synergistic effect. Summarized in Tables 2 and 3 below are preferred concentration content ranges (% w/w), and the optimal ratios thereof, of the respective components of the nanoparticles without cryopreservants or salts.

Moreover, in preferred embodiments the nanoparticles comprise the therapeutic agent Docetaxel and ApoE3 in a molar ratio of from 45-140 (ratio of molecules of Docetaxel per each recombinant ApoE3 molecule). A mass ratio of Docetaxel to ApoE3 in the nanoparticles is preferably from 1.1 to 3.3 (Docetaxel to ApoE).

TABLE 2
% w/w

| | | Choles- | | |
|---|---|---|---|---|
| Phospho- | Triglyc- | terol | Choles- | |
| lipids | erides | Ester | terol | Apo E3 |
| 2.25-8.25 | 3.75-12.1 | 0-0.6 | 0-0.9 | 0.1-01.4 |

| | | Choles- | | | Therapeutic |
|---|---|---|---|---|---|
| Phospho- | Triglyc- | terol | Choles- | | Agent |
| lipids | erides | Ester | terol | Apo E3 | (drug) |
| 5.25-8.25 | 3.75-12.1 | 0-0.6 | 0-0.9 | 0.1-1.4 | 0.3-0.9 |

TABLE 3
Optimal Ratios

| | | Choles- | | |
|---|---|---|---|---|
| Phospho- | Triglyc- | terol | Choles- | |
| lipids | erides | Ester | terol | Apo E3 |
| 35-38 | 25-60 | 0-3 | 0-4 | 0.5-7 |

| | | Choles- | | | |
|---|---|---|---|---|---|
| Phospho- | Triglyc- | terol | Choles- | | Active |
| lipids | erides | Ester | terol | Apo E | Ingredient |
| 35-38 | 26-60 | 0-3 | 0-4 | 0.5-7 | 1-10 |

G. Lipid Nanoparticle Properties and Characteristics

It has been found that nanoparticles with a phospholipid/triglyceride ratio between 0.58 and 6.4 are convenient. Specifically, the phospholipid and triglyceride components are preferably present in the nanoparticle in a ratio ranging from 5.25-8.27 (phospholipids) to 3.75-12.1 (triglycerides). Furthermore, the ratio PL/TG between 0.58 and 0.78 are helpful for maximum loading capacity of the nanoparticles. Also, nanoparticles with a PL/TG ratio of 0.67 and free cholesterol (PL: TG: EC: CL) of 39:58:1:2 are the ones that results in the highest loading capacity (percentage of encapsulation efficiency) for the active ingredient (therapeutic agent). The weight ratio of the phospholipid and triglyceride components provides a therapeutic agent encapsulation efficiency of the nanoparticles of over 90%, as determined by HPLC.

Thus, resulting in synergistic results in achieving the desirable nanoparticle characteristics. The above-described contents and ratios are critical to achieving the unexpected characteristics and properties of the nanoparticles of the invention, as furthermore evidenced by the various Examples included below.

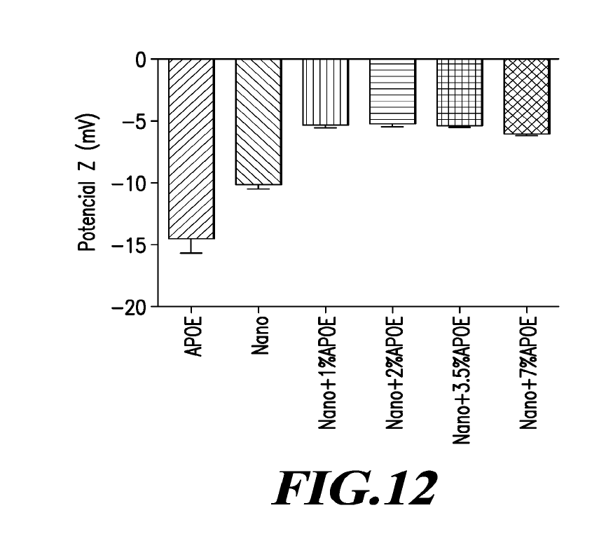

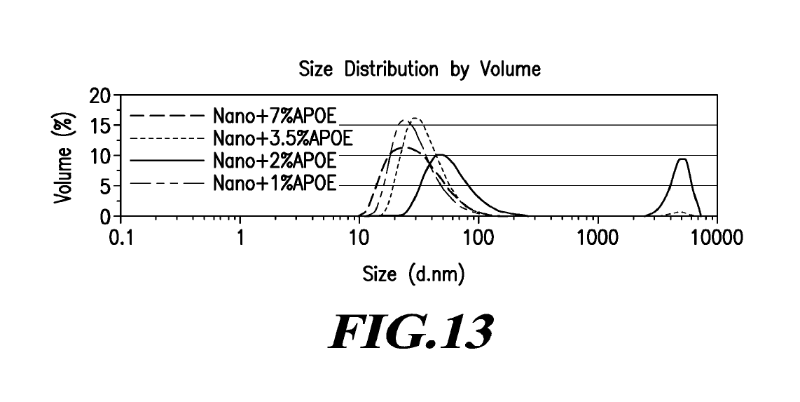

For example, it was found that varying the ratio of phospholipids/triglycerides results in changes to the charging efficiency of the nanoparticles. Specifically, lipid nanoparticles with a phospholipid/triglyceride ratio in the aforementioned ratio range exhibited the highest percentage of encapsulation efficiency for the active ingredient (85±5%). (This was determined by HPLC and based on the % of drug that was released from the nanoparticle.) Additionally, the lipid nanoparticles comprising ApoE3 demonstrated modified zeta potentials without any significant changes to the nanoparticle size (FIGS. 12 and 13).

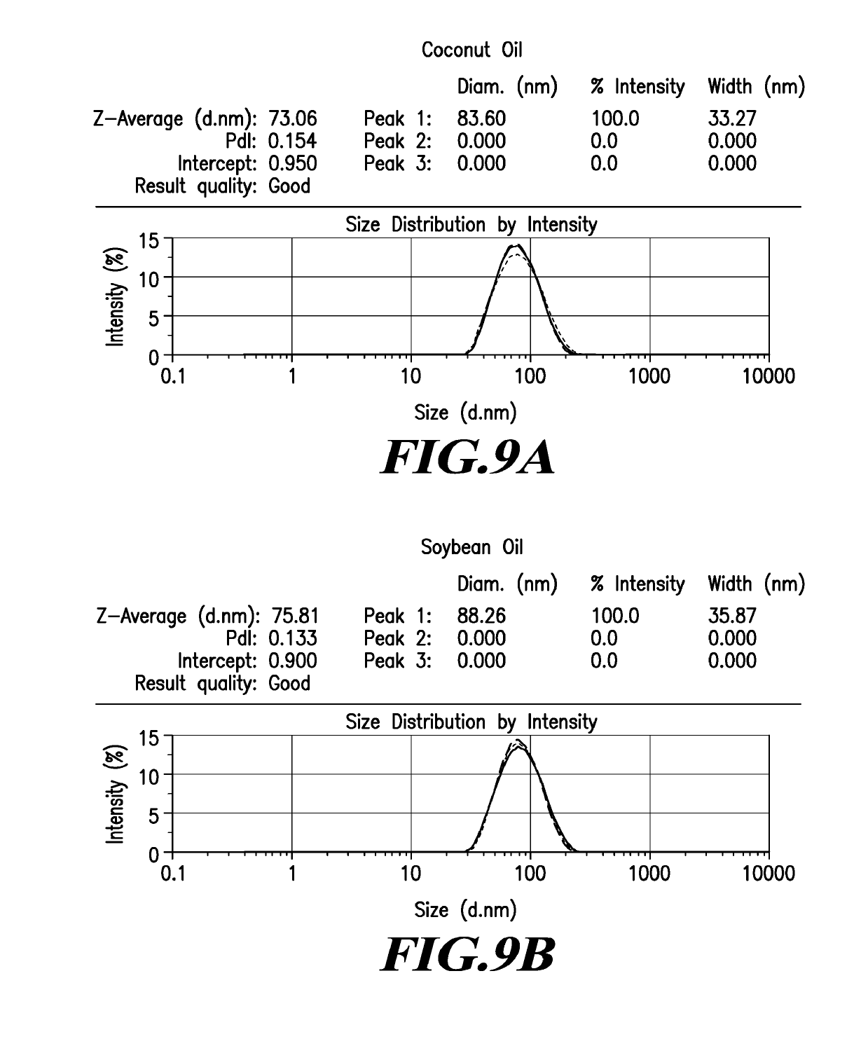

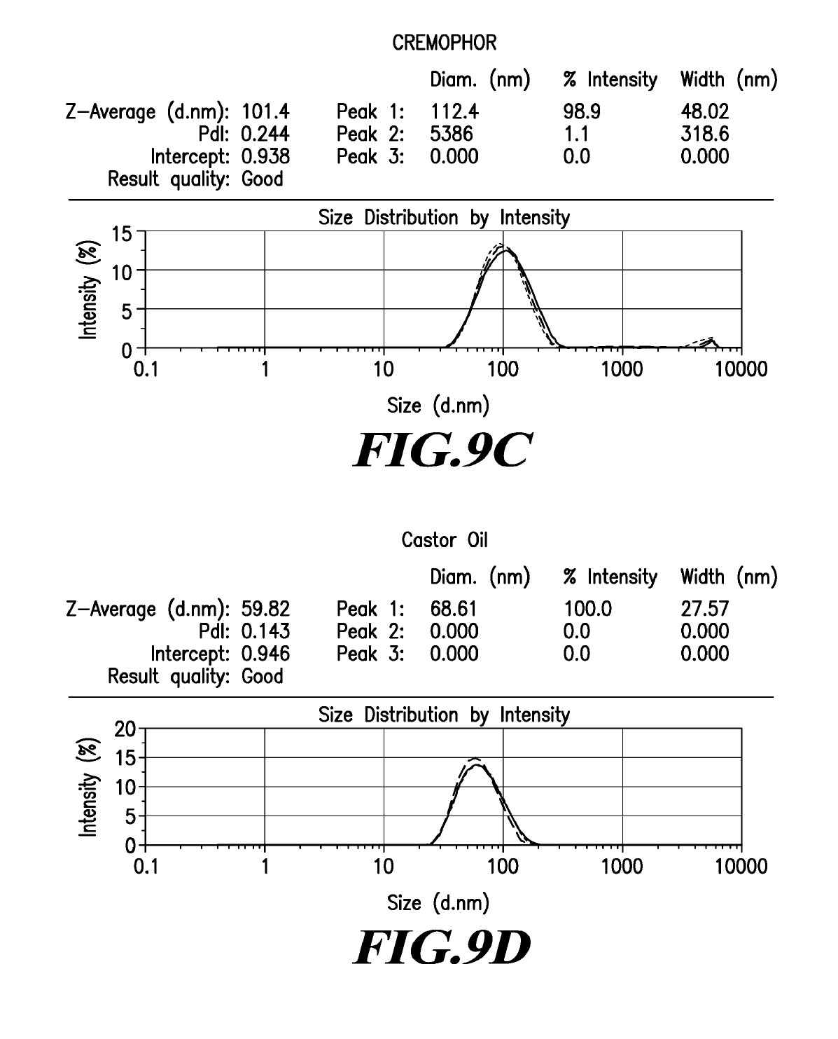

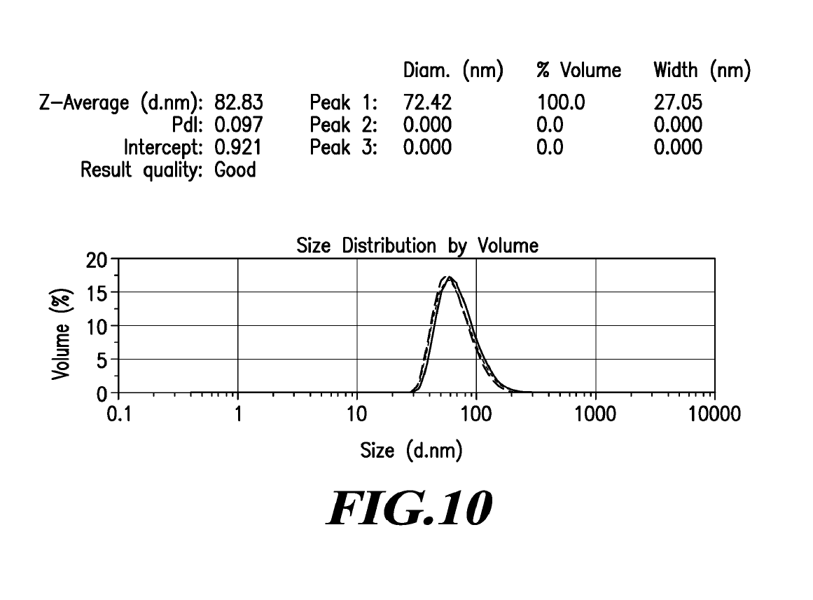

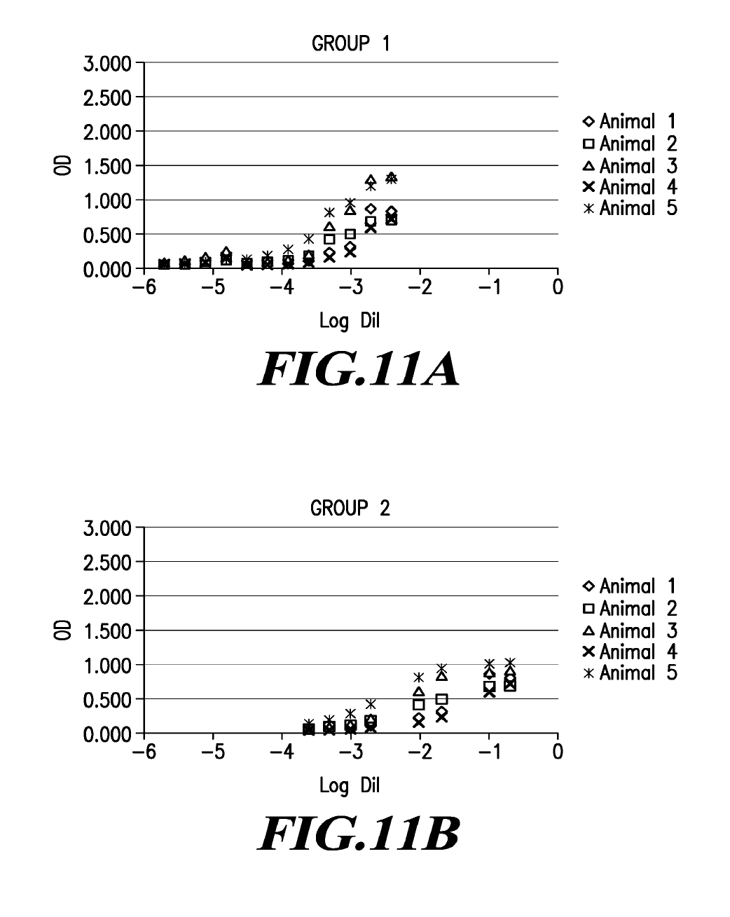

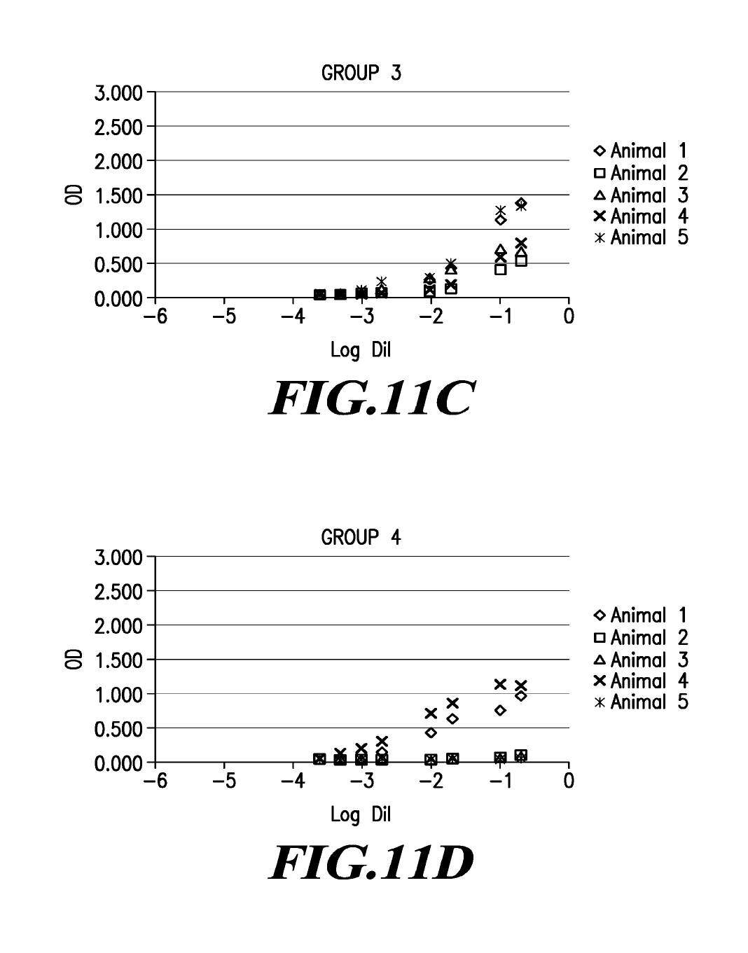

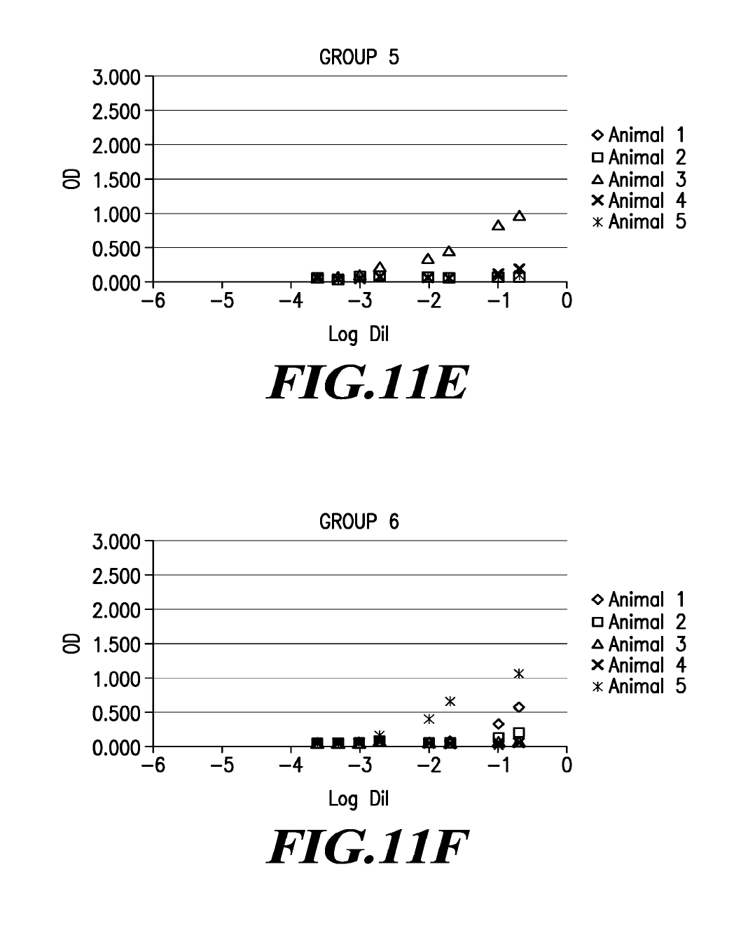

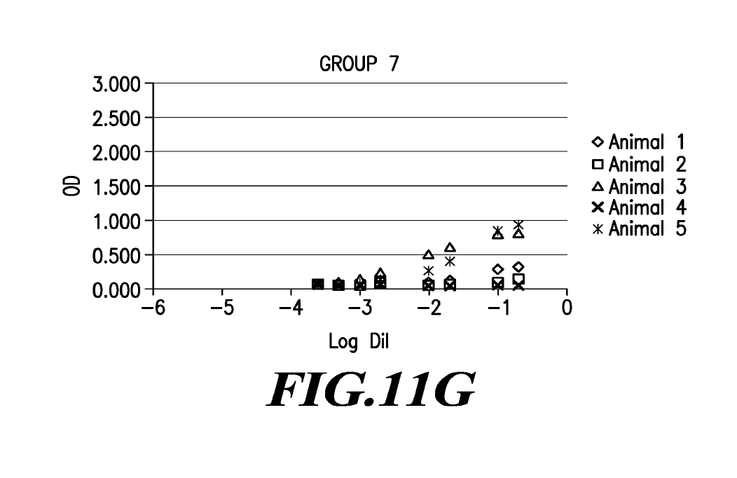

Moreover, as shown in FIG. 9, lipid nanoparticles with the same concentration for the respective components but with variations in the nature of employed triglyceride show differences both in the Z-average of the nanoparticles and dispersion (Pdi). The nanoparticles made with castor oil result in smaller particle size. Furthermore, Nanoparticles prepared with castor oil result on a more defined form (less amorphous) that can be deduced from the minor difference between the Z-average and Volume values.

In one aspect, the inventive nanoparticles may be spherical, with a size distribution range of about 20-150 nm. Also, the composition may include non-toxic surface active agents. A fundamental characteristic of nanoparticles is their instability. As particle size goes down, the interfacial area per unit mass of the dispersed system increases, and so does the interfacial energy. This increased energy will tend to drive the particles to coalescence, forming larger particles with lower energy. Extreme particle size reduction can result in significant increases in drug solubility. Materials in a nanoparticle have a much higher tendency to leave the particle and go into the surrounding solution than those in a larger particle of the same composition. This phenomenon can increase the availability of drug for transport across a biological membrane, but it can also create physical instability of the nanoparticle itself. This instability is seen in Ostwald ripening in which small particles disappear as material is transferred to large particles. The physical stability of nanoparticles may be improved by the use of appropriate surface active agents and excipients at the right levels to reduce the interfacial energy, controlling the surface charge of the particles to maintain the dispersion, and manufacturing the particles in a narrow size distribution to reduce Ostwald ripening.

The inventive nanoparticles preferably have an average size between 50 and 120 nm, a Z potential between −25 and −5 mV, and a PDI Dispersion Value between 0.08 and 0.30. In a culture with lipoprotein-free serum, the inventive nanoparticles, have a lower IC50 (inhibitory concentration 50%) and a higher selective index in cancer cells as compared to Docetaxel in its regular formulation, as demonstrated by the Examples below.

The surface active agents comprised in the inventive nanoparticles preferably include Sodium Taurodeoxicholate and Poloxamer 188—both nontoxic agents—in contrast to other conventionally used surface active ingredients, such as Polysorbate 80.

Toxicology of Intravenously administrated Poloxamer 188 indicates that its systemic toxicity is low. The intravenous LD50 was reported to be greater than 3 gm/Kg of body weight in both rats and mice. More recently, it has been described as one of the best pharmaceutical excipients for drug delivery; furthermore, it has been proven to have a neuroprotective effect once it passes through the BBB (See Domb, Abraham J., Joseph Kost, and David Wiseman, Handbook of Biodegradable Polymers, (1998); Patel, H. R. et al. (2009); and Frim, D. M et al., (2004)). On the other hand, Sodium Taurodeoxicholate is a naturally occurring surfactant (bile salt) and, thus, it is not expected to have undesirable or toxic side effects.

Regarding the ApoE ratio and concentration needed to have active drug delivery, Nelson et al. describes the use of 8-12% and a purification step, in order to eliminate all unbound proteins. According to embodiments of the invention, only 1% is needed to have the ApoE3 adsorb into the nanoparticles for targeted drug delivery. This also leads to fewer manufacturing steps to eliminate ApoE excess, thus making the manufacturing process more effective. In embodiments wherein the nanoparticles are loaded with Docetaxel, a preferred mass ratio of Docetaxel to ApoE3 in the nanoparticle is from 1.1 to 3.3 (Docetaxel to ApoE3). A molar ratio of Docetaxel molecules per each recombinant ApoE3 molecule in the nanoparticle is preferably from 45 to 140. In certain embodiments, the molar ratio of Docetaxel to ApoE3 in the nanoparticle is 126.

An additional advantage of the lipid nanoparticles includes the presence of the lipid core with a high retention capacity for liposoluble active ingredients without the need for conjugation. Although it has been mentioned in prior publications that no covalent modification of the active substance may be required for incorporation into a LDL particle, conjugation of active ingredients is common in order to keep the active ingredient inside the nanoparticle for a longer period of time, resulting in increased stability and avoidance of uptake of the active ingredient by non-targeted cells. Despite not being conjugated, in vitro tests showed that in human plasma the therapeutic agent is kept inside the lipid nanoparticles of the invention for at least 72 hours, and then transported by the nanoparticles without significant loss. Furthermore, when comparing TAXOTERE with the nanoparticles of this invention, after 72 hours, the nanoparticles showed lower release of the active ingredient when compared with TAXOTERE. As shown in Example 4, the use of these nanoparticles for target delivery results in less toxic effects of the drugs.

The stability of the lipid nanoparticles of the invention is yet another advantage over previously described LDL particles.

Unlike Nelson's product, which is stable for only 2 weeks at 4° C., stability results for compositions of nanoparticles loaded with docetaxel according to embodiments of the invention have demonstrated that the liquid formulation is stable for at least 30 days at 4° C., without significant changes in the nanoparticle size, polydispersity, Z potential and active ingredient content (assay). Also, no increase of the active ingredient impurity levels has been detected. Furthermore, a lyophilized composition according to further embodiments of the invention is stable for at least 18 months at 25° C., without significant changes in particle size, polydispersity, Z potential and active ingredient content (assay). Also, the level of impurities for the active ingredient does not increase at higher rates than what it does in the reference products.

In one embodiment, the invention refers to a lyophilized pharmaceutical composition, as well as to a reconstituted solution of the lyophilized composition. In embodiments providing for a pharmaceutical composition that has been reconstituted from a lyophilized composition, the molar ratio of Docetaxel molecules per each recombinant ApoE3 molecule in the reconstituted composition is from 45-140.

Also important is the manufacturing process, as discussed further herein. In comparing the composition of the inventive nanoparticles with various previously described nanoparticle compositions, certain clear differences include not only the specific components (ingredients) used within the structural configuration of the nanoparticle, but also the specific component ratios, and the presence of ApoE bonded to the nanoparticle surface.

The lipid nanoparticles of the invention not only structurally distinguish over previously described nanoparticles or similar artificial carriers, but also distinguish based on the unexpected properties resulting from the specific combination of components that are not achieved by previously described nanoparticles.

For example, McChesney et al. (U.S. Patent Application Publication No. 2015/0079189) describes synthetic low density lipoprotein (LDL) nanoparticles for the purpose of targeted cancer therapies These nanoparticles are comprised of a mixture including phospholipids, triglycerides, cholesterol ester, and free cholesterol, but are not coated with proteins triggering clearance processes in the tissues of the reticuloendothelial system, as previously mentioned. The nanoparticles of the invention, on the other hand, require the therapeutic agent to be dissolved in the triglyceride component (e.g., Castor Oil) in the nanoparticle core. Moreover, the lipid nanoparticles of the invention do not trigger an immunogenic response and thus allow for the use of ApoE in the formulation. As it has been shown that each individual has different levels of apolipoproteins in the body based on the varying physiological conditions of each individual, the amount of Apo proteins available results in a wide range of variability upon administration of the nanoparticles (see e.g., Liu et al., 2015). The presence of non-immunogenic ApoE3 in the nanoparticles of the invention, however, overcomes this difficulty. As demonstrated by the Examples below, the native ApoE3 does not bind or binds very poorly to the nanoparticle after intravenous injection, and the presence of ApoE3 in the nanoparticles selectively increased their targeting to cells. In this regard, the nanoparticle with ApoE3 reaches the target tissue 20% more efficiently than the nanoparticles with no attached apolipoprotein (See Example 10).

As mentioned before and shown in Example 10, the apolipoprotein is non-immunogenic. Furthermore, in embodiments, the formulation of this invention is non-immunogenic and all of its components are FDA approved, thus resulting in an innocuous formulation suitable for pharmaceutical use.

As shown in the results of Example 6, toxicity of the active ingredient is reduced when is within the nanoparticle. Drug toxicity is even lower when facing a situation of active transport to targeted specific tissues, compared to encapsulated drug without but without the Apo E3 to generate the active transportation.

Kreuter et al., in its publication titled “Apolipoprotein-Mediated Transport of Nanoparticle-Bound Drugs Across the Blood-Brain Barrier” describe a nanoparticle formulation which uses Polysorbate 80 for the attachment of ApoE. Their results suggest that the presence of Polysorbate 80 is needed in order to achieve the attachment of the ApoE to the nanoparticle. However, the inventors found and embodiments of the invention provide for the preparation of a stable nanoparticle formulation with ApoE bonded thereto without the need for Polysorbate 80. That is, the apolipoprotein component, or ApoE3, is bonded to the surface of the nanoparticle without Polysorbate 80. The toxic effects of tensoactives, such as Polysorbate 80, are well known, and the pharmacological and biological effects caused by tensoactives have been described as acute as hypersensitivity reactions, peripheral neuropathy, cumulative fluid retention syndrome, etc. That is the reason why efforts have been made to avoid the use of toxic surfactants and co-surfactants. (See Coors et al., 2005).

### III. Preparation of Nanoparticles

There are many previously described manufacturing methods of nanoparticles including: (1) high pressure homogenization, both hot and cold homogenization; (2) microemulsion-based; (3) ultrasonication, including probe and bath ultrasonication; (4) solvent evaporation; (5) solvent emulsification-diffusion; (6) double emulsion; and solvent displacement technique (7).

Most of the methods developed for producing lipid nanoemulsions are based on traditional emulsion techniques. Furthermore, the two principal methods used are the high pressure homogenization and microemulsion techniques. Hot, as well as cold, homogenization (1) processes can be used for the preparation of lipid nanoparticles; and in both the active compound is dissolved or dispersed in the melted lipid prior to homogenization step. High pressure homogenizers push a liquid using high pressure through a narrow gap (few microns). Particles formed are in submicron range due to very high shear stress and cavitation forces generated in the homogenizer. This method has as principal disadvantages the high energy input, the complex equipment required and the possible degradation of the components caused by HPH.

In microemulsion techniques (2), the melted lipid containing drug is mixed with an aqueous phase containing surfactant and co-surfactant, which is prepared at a defined temperature (high) and in such a ratio to form microemulsion. The hot microemulsion is then diluted into excess of cold water. Sudden reduction in temperature causes breaking of the microemulsion, converting it into nanoemulsion, which upon recrystallization of lipid phase produces lipid particles. Break in microemulsion is supposed to be due to the dilution with water and the reduction in temperature narrowing the microemulsion region. Microemulsion gives reduced mean particle size and narrow size distribution, the procedure is easy to scale up and does not require high energy; however, it requires a high concentration of surfactants and co-surfactants and a final step of concentration to obtain the final formulation.

For the ultrasonication method (3), including probe and bath sonication, a thin film of lipid phase is formed upon evaporation of solvent followed by ultrasonic dispersion in the presence of aqueous surfactant solution at high temperature; subsequent cooling of the system lead to the formation of lipid nanoparticles. Also, the nanoparticles may be obtained by emulsification dispersion followed by ultrasonication. Those methods require high energy input process, and give polydisperse distributions of the nanoparticles. It is also possible for metal contamination caused by the use of a probe ultrasonic.

Solvent evaporation (4) allows obtaining nanoparticles and microparticles by solvent evaporation in oil-water emulsions via precipitation. In the solvent emulsification-evaporation the lipids are dissolved in a water-immiscible organic solvent (e.g. toluene, chloroform) which is then emulsified in an aqueous phase before evaporation of the solvent under condition of reduced pressure. The lipid precipitates upon evaporation of the solvent thus forming nanoparticles. It could be possible to find organic solvent residues in the final formulation and usually a final concentration step is required.

In the Solvent emulsification-diffusion technique (5), partially water miscible solvents (e.g. benzyl alcohol, ethyl formate, tetrahydrofuran) are used to dissolve the lipids and drugs and then the resultant organic solution is quickly dispersed into an aqueous solution with some emulsion stabilizing compound. This organic phase is then emulsified in an aqueous solution containing surfactant under mechanical stirring for the preparation of an o/w emulsion. The subsequent addition of excess water to the system causes solvent diffusion into the external phase and the lipid starts precipitating. The solvent can be eliminated by ultra-filtration or by distillation. After the removal of the organic solvent, an aqueous dispersion of lipid nanoparticles is formed. The disadvantage of this method is the use of organic solvent (and this possible residues in final formulation) and the lack of large scale production caused by the low encapsulation by the use of partially water miscible solvents.

Multiple (or double) Emulsion Technique (6), this is a modified solvent emulsification-evaporation method based on a w/o/w double emulsion. The first step of emulsification is followed by solvent evaporation. The drug is encapsulated with a stabilizer to prevent drug partitioning to external water phase during solvent evaporation in the external water phase of w/o/w double emulsion. Although it allows the incorporation of hydrophilic drugs, the nanoparticles have a large particle size in the final formulation.

In the solvent Displacement technique (7), a solution of the lipid in a water-miscible solvent or a water-miscible solvent mixture is rapidly injected into an aqueous phase with or without surfactant. In this process, an o/w emulsion is formed by injecting organic phase into the aqueous phase under constant stirring. The oil phase is a semi-polar water-miscible solvent, such as ethanol, acetone or methanol, lipid material is dissolved in it and then the active compound is dissolved or dispersed in this phase. In this procedure solvent displacement of diffusion takes place and lipid precipitate is obtained. Solvent removal is necessary and can be performed by distillation. The lipid nanoparticles are formed after evaporation of the water miscible organic solvent. Particle size is dependent on the preparation conditions such as amount to be injected, concentration of lipid and emulsifier. The disadvantage of this method may be the possible organic solvent residues in the final formulation. (See Sunil Prakash Chaturvedi et al., 2012; Beatriz Lasa-Saracihar et al., 2012; and Hu, Fu-Qiang et al., 2006.)

All of the foregoing methods are standard procedures for the preparation of lipid nanodispersions. In general, these methods have good reproducibility and some of them have well established large-scale homogenization technology. However, the preparation of lipid nanodispersions with these conventional methods involves several critical process parameters, such as high temperatures, high pressures, toxicologically problematic solvents, etc., and in many cases complex equipment is required. Also, these extreme conditions could generate degradation of the components of the nanoparticles as well as the Active Substance.

It is for the above-mentioned reasons that a new manufacturing method is provided herein, which is scalable and controllable, pharmaceutically acceptable, with low energy requirements and provides monodisperse and stable nanoparticles formulations.

The present invention describes a new manufacturing procedure to obtain the nanoparticle formulation which offers clear advantages over the previously described methods such as, the use of only pharmaceutically acceptable and FDA approved components, easy handling and scalable without the need of sophisticated equipment. This procedure allows obtaining particles with mean size of 100 nm; stable and suitable for pharmaceutical purposes with yields and efficiencies of 100%.

The method can be considered as a low energy process since the nanoemulsion is spontaneously formed, triggered by the rapid diffusion of the surfactant and solvent molecules (dispersed phase) in to the continuous phase. In contrast to what Schubert et al. describes injection velocities employed, the lipids and the surfactants used in this invention, do not generate precipitation by local supersaturation and consequently avoids the appearance of large particles that should be filtered later, allowing to obtain 100% efficiency.

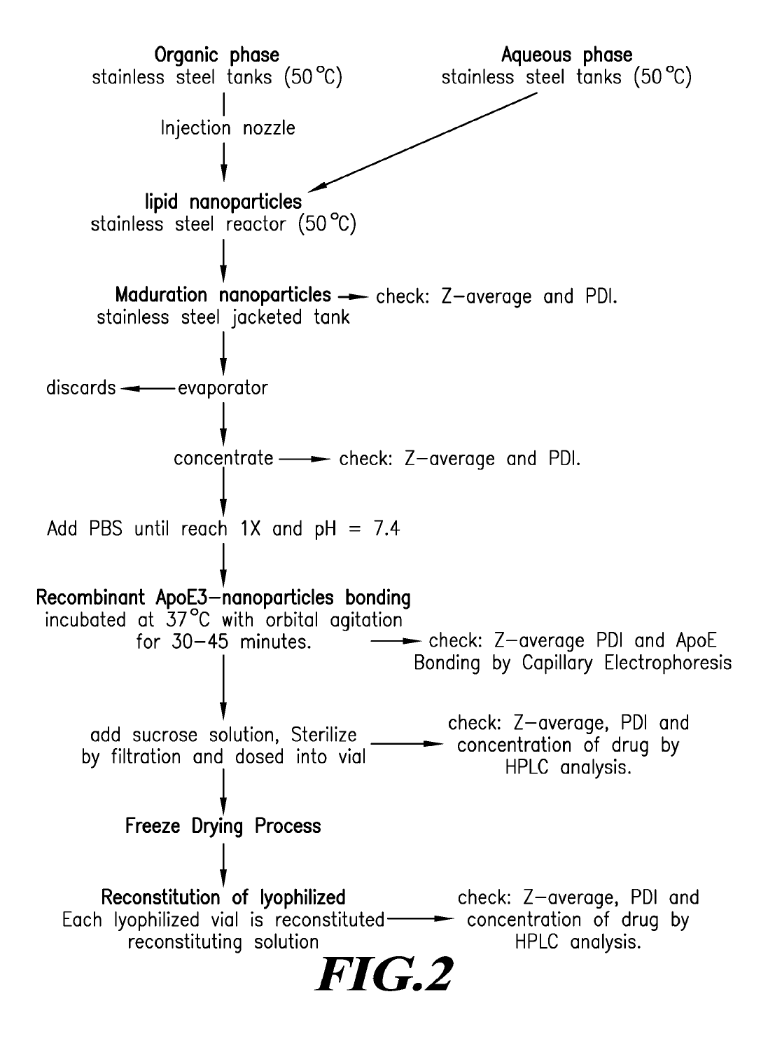

As depicted by the flow chart in FIG. 2, the manufacturing procedure consists of: (1) combining the lipophilic active ingredient, phospholipids and triglycerides to form a mixture (Organic Phase); (2) combining water for injection and the surfactants to obtain the aqueous phase; and (3) injecting the organic phase at 1-1.5 mL/sec. into the aqueous phase heated at 30-50° C. through an injection nozzle in a highly turbulent regime to obtain the nanoparticles with an average size between 20-150 nm. In embodiments, the manufacturing method includes concentrating the obtained lipid nanoparticles to the appropriate concentration of total lipids as described herein, and adding ApoE3 in an aqueous solution at pH 7.4 at around 37° C. to the obtained nanoparticles to coat the nanoparticles. The method may further include adding sucrose to obtain a composition suitable for lyophilization and lyophilizing the composition.

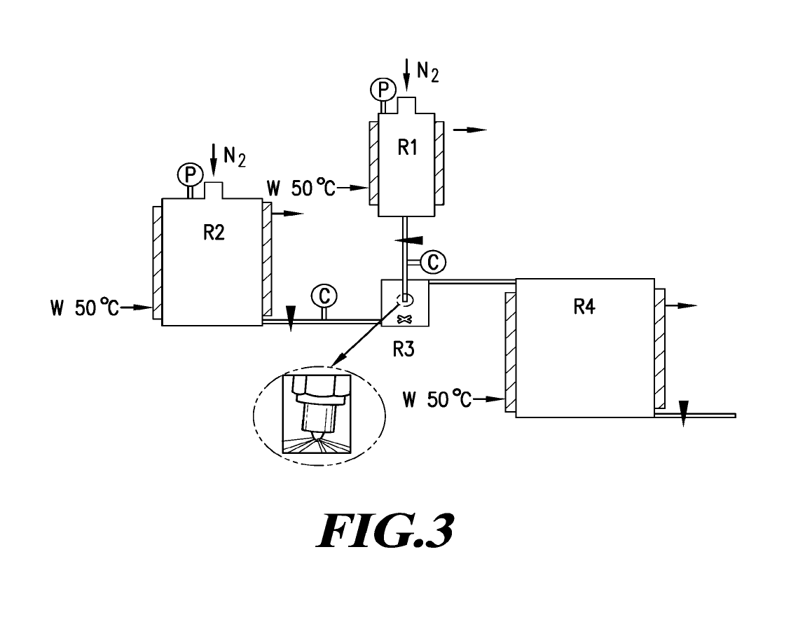

In one embodiment, the manufacturing method described herein involves the use of a system as shown in FIG. 3. In this exemplary equipment (R=reservoir, P=barometer, C=flowmeter, W=water at 25-50° C.), two stainless steel tanks (R1 and R2) with a 20-60° C. thermostatized jacket and able to resist a pressure 40 to 200 atmospheres; are connected at the top to a nitrogen tube. The R1 tank has a steel pipe welded to a direct injection nozzle at its bottom portion, which has one-four holes that are each 200-800 microns in size. The injection nozzle is inserted from the top towards a central portion of another smaller stainless steel reactor (R3). R2 is connected to R3 by a steel pipe. Finally R3 is connected to a fourth stainless steel jacketed tank (R4) that has a tube evaporator communicating exit which has two fraction containers, one for discards and the other for collecting the concentrate.

In another embodiment the present invention relates to a method of preparing nanoparticles comprising: (1) dissolving the Active Ingredient in the lipid components (preparation of an organic-oil-phase) at 20-50° C. in a stainless steel reactor pressurized to 50-1400 atmospheres; (2) injecting the oil phase into a 4-hole (200 microns each) injector at a flow rate of 22 cm³/sec and a linear velocity of 177 m/s; (3) generating the nanoemulsion by the collision of the oil phase with a aqueous phase flow of 88 cm³/sec; generating a very fine spray; and (4) keeping the obtained nanoemulsion at 20-40° C. for 0.5-3 hours with constant stirring.

In another embodiment, the aqueous phase is maintained at 20-60° C. inside the reactor R2. In preferred embodiments, the surfactants in the aqueous phase are choline taurodeoxycholate and Poloxamer 188. In another embodiment, the nanoemulsion is obtained by the collision of the oil phase at a flow rate of 22 cm³/sec and with the aqueous phase flow of 88 cm³/sec in R3. In 10-20 minutes, the mixture generated in R3 becomes a clear colloidal lipid nanoparticle solution.

In the aqueous phase, the process generates lipid nanoparticles with entrapped therapeutic agent (drug) containing 20% ethanol and surfactants. The solution is then concentrated by distillation under reduced pressure, or evaporation under reduced pressure at 25 mmHg (bath temperature of 40-50° C.) in a tube evaporator, to reduce its volume. The lipid nanoparticles obtained by the above process were found to have a Z-average between 20-100 nm (measured by DLS), PDI less than 0.2 (measured by DLS), zeta potential of about −25 to −45 mV, and turbidity of 600-900 NTu.

In one embodiment, a phosphate buffered saline is added to the concentrated colloidal liquid nanoparticle solution at room temperature resulting in a pH 7.4 solution.

In another embodiment, a 1-2 mg/ml solution of human recombinant ApoE3 is added to the lipid nanoparticles formulation (final concentration of 1% of ApoE3) and the solution is incubated at 37° C. for 30-60 minutes under constant orbital agitation.

In one embodiment, the formulation is then sterilized by membrane filtration (0.22 μm), dosed into suitable clean and sterile vials, lyophilized, sealed, and stored at room temperature for at least 12 months.

Although the inventive process employs highly turbulent conditions, it is considered to be a low-energy process due to the nano-emulsion formation being triggered by the rapid diffusion of surfactant and/or solvent molecules from the dispersed phase to the continuous phase.

An advantage of the manufacturing process described herein is that it is both scalable and controllable, thus allowing it to be easily used in a pharmaceutical plant and under GMP conditions. Furthermore, the process produces monodispersed nanoparticles smaller than 100 nm without the need to undergo high pressure homogenization, high speed homogenization, or size reduction ultrasonication.

### IV. Compositions Comprising Lipid Nanoparticles

Included within the scope of the invention are formulations comprising at least one nanoparticle for human or veterinary use, such as pharmaceutical compositions. Such compositions may further comprise pharmaceutically-acceptable carriers or excipients, optionally with supplementary medicinal agent. In embodiments, the pharmaceutically-acceptable excipient is selected from the group consisting of sucrose, sodium taurodeoxycholate, Poloxamer 188, sodium acyl phosphate, potassium dihydrogen phosphate, sodium chloride and potassium chloride. Conventional carriers, such as glucose, saline, and phosphate buffered saline, may also be used in such compositions.

In embodiments, the compositions may contain pharmaceutically acceptable excipients as required to approximate physiological conditions, such as pH adjusting and buffering agents, tonicity adjusting agents and the like. Other ingredients which may be included in the pharmaceutical compositions of the invention are known in the art and described in, e.g., Genaro, Remington's Pharmaceutical Sciences, Mack Publishing Co., Easton, Pa., (1985).

Concentrations of the lipid nanoparticles in compositions within the scope of the invention can vary widely, such as from less than about 0.3% or at least about 1%, to as much as 5-10% by weight.

### V. Kits

Embodiments of the invention relate to kits comprising the lipid nanoparticles and compositions described herein. Such kits may contain a lyophilized preparation of the nanoparticles and a sterile aqueous solution for mixing prior to administration.

### VI. Therapeutic Methods

The lipid nanoparticles may be administered to a subject in need of treatment to effectively deliver active agents to the targeted tissue. In therapeutic application, an effective amount of drug-containing lipid nanoparticles can be administered to a subject by any mode allowing the nanoparticles to be taken up by capillary endothelial cells. That is, delivery of the active agents to target tissues is by an active receptor-mediated process known as transcytosis.

Provided are methods for treating cancer in a patient, comprising administering a pharmaceutical composition comprising a therapeutically effective dose of the nanoparticles described herein. In preferred embodiments, the nanoparticles of the composition are loaded with Docetaxel and are administered to treat lung cancer or colon cancer in the patient.

It will be appreciated that that the effective amount of the lipid nanoparticles, as well as the route or mode of administration of the nanoparticles (and/or the therapeutic agent encapsulated in the nanoparticles) may vary according to the nature of the therapeutic agent to be administered or the condition to be treated. The specific dosage to be administered is of an amount deemed safe and therapeutically effective for the particular patient under the particular conditions and may be dependent on the mode of administration thereof. The modes of administration may include (but are not limited to) oral, intravenous, intramuscular, subcutaneous, transmucosal, and transdermal. In preferred embodiments, a composition comprising the nanoparticles described herein may be administered parenterally or intravenously.

In certain embodiments, the lipid nanoparticles may be formulated for controlled release, such that the release of the therapeutic agent from the nanoparticle is maintained to achieve the desired therapeutic level of the therapeutic agent in blood or tissue for an extended period (hours or days).

In still other embodiments, the invention provides a method of treatment that includes administering a therapeutically effective amount of a therapeutic agent enclosed in the lipid nanoparticles, whereby the lipid nanoparticles of the invention may include a targeting function due to the attachment of ApoE3. Targeting is a major advantage in, e.g., treatments of malignant tissues that have shown to have enhanced receptor expression, due to the favored uptake of a therapeutic agent encased in the nanoparticles. Furthermore, certain therapeutic agents, when encapsulated in the nanoparticles, may be used to target the necessary tissue (e.g., kill cancer cells or tumors more effectively) than the free drug, while reducing the impact the drug would otherwise have on normal tissues.

Therapeutic methods of the invention may include methods for treatment of cancer, such as leukemia, neuroblastoma, glioblastoma, cervical, colorectal, pancreatic, renal melanoma, lung, breast, prostate, ovarian, head and neck. Preferred therapeutic methods of the invention include methods for treatment of cancer tissues associated with over-expression of r-LDL, such as lung and prostate cancer. Specifically, the invention relates to methods of cancer therapy, comprising treating cancer tissue with the nanoparticles of the invention that are loaded with and deliver effective dosages of Docetaxel via r-LDL-mediated endocytosis.

While the invention has been described with respect to particular embodiments, it will be apparent to those skilled in the art that various changes and modifications may be made without departing from the spirit and scope of the invention defined in the appended claims. Such modifications are also intended to fall within the scope of the claims. Persons skilled in the art would recognize that there exist a broad range of possible clinical applications of the lipid nanoparticles of the invention.

### EXAMPLES

The following Examples serve to further illustrate specific embodiments and are not to be construed as limiting the scope of the invention in any way.

### Example 1: Nanoparticles Manufacturing

### Organic Phase Preparation

140 g of anhydrous ethanol, 0.805 g of egg yolk PL (Egg PC 80 lipoid), 0.042 grams of Docetaxel USP, 1.224 grams of Castor oil USP, 0.028 grams of Cholesteryloleate and 0.042 grams of cholesterol were added into a 250 ml round-bottomed flask inside a thermostatized bath with bubbling nitrogen previously heated to 40° C. The solution was stirred until complete dissolution of all components.

### Aqueous Phase Preparation

560 g of WFI (previously filtered with a 0.45 μm PVDF membrane), 0.14 g of Lutrol F68 and 0.07 g of choline taurodesoxycholate were added to a 1 L glass Schott bottle inside a thermostatized bath with bubbling nitrogen previously heated to 40° C. The mixture was stirred with a 60 mm stirring bar at 500 rpm.

### Nanoparticle Manufacture

To obtain the lipid nanoparticles, organic phase was injected into the aqueous phase (heated at 40° C. and stirred at 500 rpm) at a rate of 1-1.5 ml/sec using a 4-hole nozzle. The mixture was stirred at 250 rpm for 45 minutes. At this point, the size (Z-average) and dispersion (PDI) of the newly formed nanoparticles was measured as a process control before continuing on with the manufacturing process. Then, the nanoparticles were concentrated by distillation under reduced pressure until the desired fat percentage value was reached. After concentrating the nanoparticles, reconstituting solution was added until a 1× concentration and a 7.4 pH of the solution was reached.

### Recombinant ApoE3 Bonding to Nanoparticles

A 2 mg/ml ApoE3 solution (in phosphate buffer) was added to a 500 ml round bottom flask containing the produced nanoparticle solution (20 mg/ml of total lipid content loaded with Docetaxel) until reaching a final concentration of 0.26 mg/ml ApoE3 in the solution. The resulting solution was then incubated at 37° C. with orbital agitation for 30-45 minutes. The size (Z-average) and dispersion (PDI) of the resulting nanoparticles was then measured a process control.

### Example 2: Freeze Drying of Nanoparticle Formulation

A 60% w/w sucrose solution was added to the round bottom flask containing the mixture of recombinant ApoE3-bonded nanoparticles obtained according to Example 1 until a final concentration of 11% sucrose was reached. The solution was sterilized by filtration with a PVDF 0.22 μm membrane, with the integrity of the filter being checked before and after filtration. The solution obtained under these conditions was checked by HPLC analysis to have a final Docetaxel concentration of 0.6 mg/ml. To obtain 1.8 mg of Docetaxel in each vial, approximately 3 ml of the solution were dosed into each 10 ml vial.

The vials were pre-stopped using 20 mm bromobutyl lyophilization stoppers (Datwyler Pharma Packaging FM357/1 Grey), placed inside the lyophilization chamber, and freeze dried according to the following cycle (Table 4):

TABLE 4
Lyo Cycle

| Step | Vacuum | Step Duration (hh:mm) | Temperature (° C.) |
|---|---|---|---|
| 1 | No | 15:00 | −50 |
| 2 | Yes | 5:00 | −50 |
| 3 | Yes | 24:00 | −35 |
| 4 | Yes | 3:00 | 0 |
| 5 | Yes | 36:00 | 25 |

Once the freeze drying cycle was completed, the vacuum was released with sterile nitrogen and the vials were stoppered inside the lyo machine. Finally, the vials were sealed with 20 mm aluminum seals (West Pharmaceutical Services), checked by visual inspection, and stored. Each of the stored vials ultimately contained 100 mg of lipid nanoparticles with 1.8 mg of Docetaxel, 1 mg ApoE3, and 11 mg of sucrose.

### Reconstituting Solution Preparation and Resuspension

In a sterile area using aseptic procedures, 103 mg of sodium acid phosphate USP, 18 mg of potassium dihydrogen phosphate USP, 725 mg Sodium Chloride USP and 20 mg of potassium chloride USP were added into a 1000 ml round bottomed flask containing 250 ml of WFI. The solution was magnetically stirred until complete dissolution, and the homogeneous solution sterilized by filtration using 0.22 μm PFVD filters, with the integrity of the filters being checked prior and after filtration.

Following filtration, 5 ml of the solution was dosed into each 10 ml sterile vial. The vials were closed using 20 mm bromobutyl stoppers for solutions (West Pharmaceutical Services, cod: 10145138) and sealed with 20 mm aluminum seals (West Pharmaceutical Services). Finally, the vials of reconstituting solution were sterilized by autoclaving at 120° C. for 20 minutes, checked by visual inspection, and stored at room temperature.

As described further herein, 3.0 ml of the reconstituting solution was used to reconstitute each lyophilized vial of nanoparticles.

### Exemplary Nanoparticle Formulation

As described herein, the inventive nanoparticles comprise: phospholipids (PL), triglycerides (TG), cholesterol (C), cholesteryl ester (CE), and ApoE3. Presented in Table 5 below is an exemplary formulation of the nanoparticles according to an embodiment of the invention.

TABLE 5

| | | |
|---|---|---|
| Therapeutic or Image Agent | 1-2 | mg |
| EpoE3 | 0.66 | mg |
| Egg Yolk PL (USP) | 25.31 | mg |
| Castor Oil (USP) | 38.52 | mg |
| Cholesterol (USP) | 1.32 | mg |
| Cholesteryl Oleate | 0.88 | mg |
| Sucrose (USP) | 324.00 | mg |
| Na TDC | 2.22 | mg |
| Poloxamer 188 (USP) (Lutrol F68) | 4.44 | mg |
| Sodium Acid Phosphate (USP) | 2.48 | mg |
| Potassium Dihydrogenphosphate (USP) | 0.43 | mg |
| Sodium Chloride (USP) | 17.41 | mg |
| Potassium Chloride (USP) | 0.47 | g |

The aforementioned formulation provided for nanoparticles within the scope of the invention having monodisperse behavior, uniform particle size, and high drug entrapment efficiency. An advantage of this type of formulation is the ability to be lyophilized and then reconstituted, without losing any of the aforementioned advantageous physicochemical characteristics.

### Exemplary Restorative Solution

Presented in the Table 6 below is an exemplary formulation of a restorative solution for use according to embodiments of the invention.

TABLE 6
Restorative Solution

| | | |
|---|---|---|
| Sodium Acid Phosphate (USP) | 1.72 | mg |
| Potassium Dihydrogenphosphate (USP) | 1.61 | mg |
| Sodium Chloride (USP) | 12.08 | mg |
| Potassium Chloride (USP) | 0.330.20 | mg |
| WFI | e.q. 5 | ml |

### Example 3: Nanoparticles Size and PDI

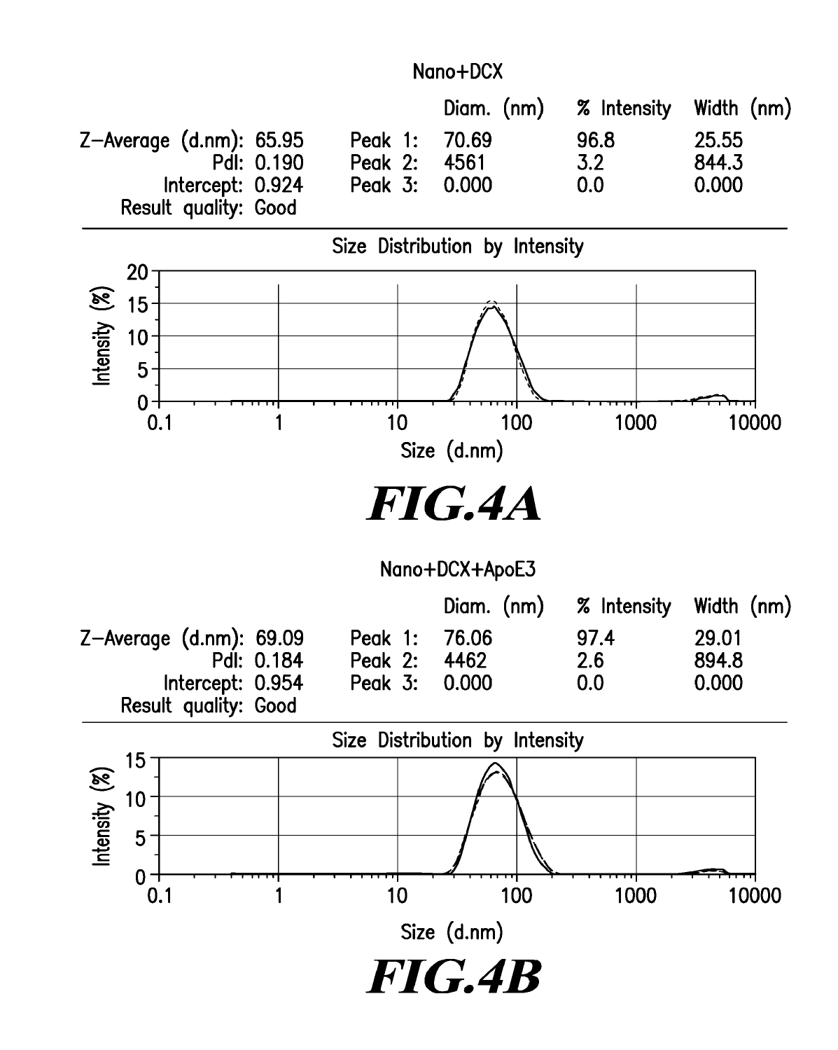

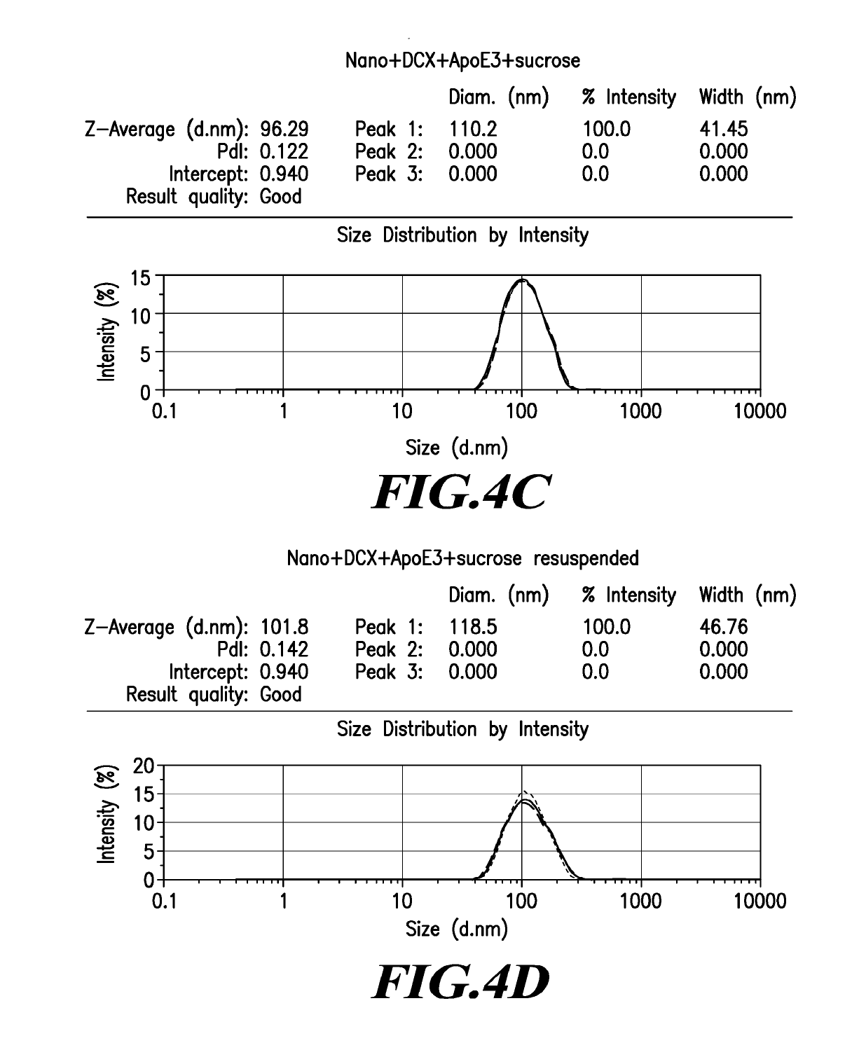

The size of the nanoparticles was determined using dynamic light scattering (DLS), and measured before and after the nanoparticles were subjected to a freeze drying process (with sucrose). The DLS results provided in FIG. 4 show the volume distribution of: lipid nanoparticles with Docetaxel (FIG. 4A); lipid nanoparticles with Docetaxel and loaded with ApoE3 (FIG. 4B); lipid nanoparticles before the freeze drying process (FIG. 4C); and the lipid nanoparticles after the freeze drying process, lyophilized and resuspended in restorative solution (FIG. 4D).

As shown in FIG. 4, no significant differences in size were observed between nanoparticles loaded with recombinant ApoE3 and nanoparticles without the recombinant ApoE3. Similarly, no significant differences in particle size were observed before versus after the drying process.

### Example 4: Nanoparticles With Different Types of Triglycerides

Four batches of lipid nanoparticles were manufactured according to Example 1 where the only variation was the type of triglyceride used. They were used: coconut oil, soybean oil, castor oil and CREMOPHOR®.

From there was measured data for the whole nanoparticle ripening for 15 minutes and then of the nanoparticle concentrated to 20 mg/ml of total lipids.

The results obtained are shown in Table 7 below:

TABLE 7

| | N253 TG COCO OIL | | N254 TG SOJA OIL | | N255 CREMOPHOR | | N256 TG CASTOR OIL | |
|---|---|---|---|---|---|---|---|---|
| | Intact | Concentrated | Intact | Concentrated | Intact | Concentrated | Intact | Concentrated |
| | Np | Np 2% lipids | Np | Np 2% lipids | Np | Np 2% lipids | Np | Np 2% lipids |
| Z-average | 160.3 | 72.5 | 164.9 | 75.7 | 171 | 96.8 | 117.7 | 58.9 |
| % Relative | 272 | 123 | 280 | 129 | 290 | 164 | 200 | 100 |
| to the lower | | | | | | | | |
| value | | | | | | | | |
| Pdi | 0.184 | 0.145 | 0.144 | 0.131 | 0.188 | 0.239 | 0.099 | 0.136 |
| volume | 217.4 | 59.6 | 207.5 | 62.7 | 668.9 | 1581.3 | 118.3 | 47.7 |
| Dif Z- | 57.1 | 12.9 | 42.6 | 13 | 497.9 | 1484.5 | 0.6 | 11.2 |
| average - | | | | | | | | |
| volume | | | | | | | | |

From these results we can see that Lipid Nanoparticles with the same formulation but with variations in the type of employee triglyceride showed differences both in the z-average of the nanoparticles and dispersion (Pdi) thereof resulting in smaller nanoparticles those made with Castor oil.

Further more lipid nanoparticles have less difference between the Z-average and volume could be considered more stable. In our case this minor difference is also attributed to the nanoparticles prepared with Castor Oil.

### Example 5: Stability of the Lyophilized Nanoparticle Formulations

After preparing the lyophilized nanoparticle formulation according to the method described herein, a stability study was carried out after two months at 25° C. This study evaluated the following variables, the results of which are summarized in Table 8 below: lyophilized appearance, appearance of the reconstituted solution, particle size distribution and zeta potential, Docetaxel assay, Docetaxel-related substances, pH, pass through needle, % water content (KF), air tightness, and endotoxins.

TABLE 8
Stability Results

| | | | Time (weeks) | | | | |
|---|---|---|---|---|---|---|---|
| Analysis | | Limits | 0 | 1 | 2 | 4 | 8 |
| Visual | Macroscopic | White powder with | Meets | Meets | Meets | Meets | Meets |
| Aspect | | different areas of pale | | | | | |
| | | yellowish tint or surface | | | | | |
| | | foreign particles | | | | | |
| | Microscopic | Amorphous crystals | Meets | Meets | Meets | Meets | Meets |
| Appearance of | | Opalescent reconstituted | 752.9 | 789.5 | 748.0 | 761.2 | 769.3 |
| Reconstituted Solution | | solution between 650 and | | | | | |
| Clarity and Degree of | | 900 NTU | | | | | |
| Opalescence, and Degree | | Reconstituted solution has | Meets | Meets | Meets | Meets | Meets |
| of Coloration | | a small yellowing. | | | | | |
| Particle Size Distribution | | Z-average: 40-100 nm | 88.21 | 82.33 | 89.57 | 93.62 | 87.43 |
| and Z-potential (DLS) | | PDI: 0.05-0.250 | 0.148 | 0.163 | 0.152 | 0.148 | 0.148 |
| | | PZ: −10 a-20 mV | 12.5 | 11.75 | 12.5 | 12.5 | 12.5 |
| Docetaxel Identification | | Retention time (RT) of | 14.026 | 14.318 | 14.638 | 14.116 | 14.080 |
| (HPLC) | | main peak of sample | | | | | |
| | | corresponds to RT of main | | | | | |
| | | peak of standard | | | | | |
| Assay (HPLC) | | 1.6-2.0 mg Docetaxel/vial | 1.776 | 1.773 | 1.774 | 1.771 | 1.772 |
| | | 95.0-105.0% of labeled | | | | | |
| | | amount | | | | | |
| Docetaxel- | 10-oxo- | NMT 0.5% | -ND | ND | ND | ND | ND |
| Related | docetaxel | | | | | | |
| Substances | (TRR = 1.17) | | | | | | |
| (HPLC): | 7-epi- | NMT 0.5% | 0.0234 | 0.0315 | 0.0228 | 0.0258 | 0.0269 |
| | docetaxel | | | | | | |
| | (TRR = 1.53) | | | | | | |
| | 7-epi-10-oxo- | NMT 0.6% | ND | ND | ND | ND | ND |
| | docetaxel | | | | | | |
| | (TRR = 1.65) | | | | | | |
| Specified | Impurities 1 | NMT 0.7% | ND | ND | ND | ND | ND |
| Unidentified | (RRT = 0.24) | | | | | | |
| Impurities | Impurities 2 | NMT 3.6% | ND | ND | ND | ND | ND |
| and Max | (RRT = 0.6) | | | | | | |
| Impurities | Total | NMT 2.5% | 0.0234 | 0.0315 | 0.0228 | 0.0258 | 0.0269 |
| | Impurities | | | | | | |
| pH | | 7-8 | 7.38 | 7.36 | 7.42 | 7.46 | 7.39 |
| Pass Through Needle | | Passes through 31 | Pass | Pass | Pass | Pass | Pass |
| | | gauge needle | | | | | |
| Water Content (KF) | | NMT 3.0% | 1.8 | 1.8 | 2.0 | 1.9 | 1.7 |
| Particulate | Ave. no. | NMT 6000 particles/vial | ND | ND | ND | ND | ND |
| Pollution: | particles ≥ | | | | | | |
| sub-visible | 10 μm | | | | | | |
| particles | Ave. no. | NMT 600 particles/vial | ND | ND | ND | ND | ND |
| (2.9.19 EP/ | particles ≥ | | | | | | |
| USP <788>), | 25 μm | | | | | | |
| Method I | | | | | | | |

| | | | | | | |
|---|---|---|---|---|---|---|
| Removable Volume (pool | Σ₅ units ≥ 3.0 ml | 3.02 | 3.05 | 3.08 | 3.06 | 3.10 |
| of 5 units)(EP 2.9.17/ | | | | | | |
| USP <1>) | | | | | | |
| Integrity (tightness) of | Samples do not show | Without | Without | Without | Without | without |
| Vials | absorption maximums near | Peaks | peaks | peaks | peaks | peaks |
| | to 660 nm | | | | | |
| External Sterility | Sterile | Sterile | ND | ND | ND | Sterile |
| Endotoxins (USP) | <1.94 EU/mg Docetaxel | 0.5 | ND | ND | ND | 0.5 |

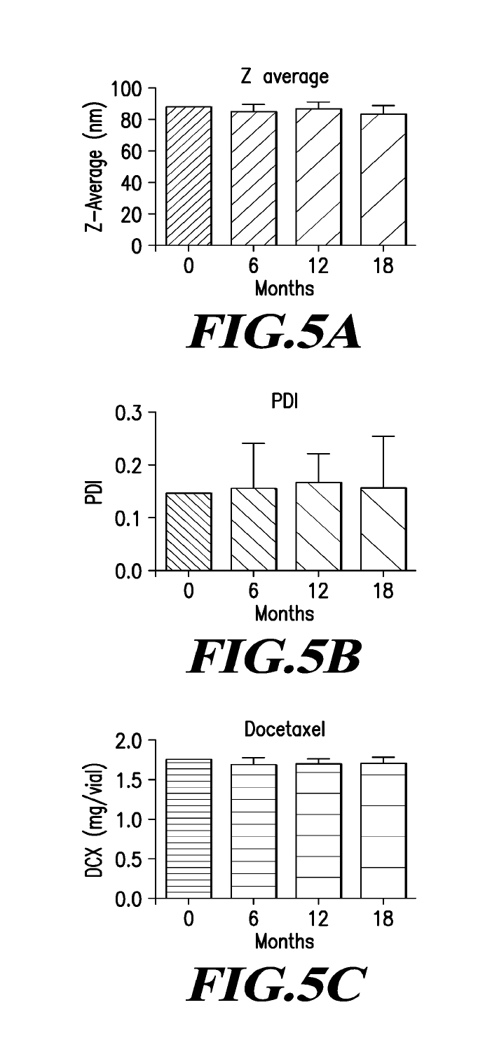

As demonstrated by the results in Table 8, the lyophilized formulation according to embodiments of the invention was stable after 2 months at 25° C. storage conditions. A longer stability assay was further carried out at 25° C. for a period of up to 18 months to measure the active content and size distribution. The stability results of this additional study are reported in FIG. 5, showing the Z-average, PDI, and Docetaxel content after 5, 12, and 18 months at 25° C. These results further demonstrate that the nanoparticle formulation according to the invention is stable for at least 18 months, during which time the nanoparticles maintain their size, polydispersity index, and Docetaxel content, thus avoiding epimerization thereof in formulation.

### Example 6: Pharmacokinetics of Docetaxel in its Formulation with Polysorbate 80 vs. Docetaxel in Lipid Nanoparticle with ApoE3 According to Invention

Changes in plasma drug concentration reflect changes in drug concentrations at the receptor site, as well as in other tissues. This study focused on determining the pharmacokinetic behavior of Docetaxel (DCX) in young New Zealand rabbits of approximately 3.5 kg, and comparing the lipid nanoparticles loaded with Docetaxel and ApoE3 according to the invention with conventional Docetaxel formulated with Polysorbate 80.

The formulations were administered intravenously at doses equivalent to 2.5 mg/kg of DCX in each. Four groups (A, B, C, and D) of 5 animals each were inoculated, with Groups A and B being treated with DCX (TAXOTERE) and Groups C and D with lipid nanoparticles of the invention (Nano+DCX+ApoE3). Blood samples were taken at 0.5, 2, 8, and 24 hours from each animal in Groups A and C, and at 1, 4, 12, and 32 hours for each animal in Groups B and D. Then, the samples were pre-treated for analysis—the proteins were precipitated with acetonitrile and then extracted with a solid phase (SPE), evaporated, and resuspended for analysis. The Docetaxel concentrations were determined by liquid chromatography coupled to mass spectrometry (using a Shimadzu UFL XR liquid chromatograph, coupled to a AB Sciex 3200 Q Trap mass spectrometer). Any adjustment of the experimental data was performed by weighted nonlinear regression of at least squares using a bi-exponential descriptive model.

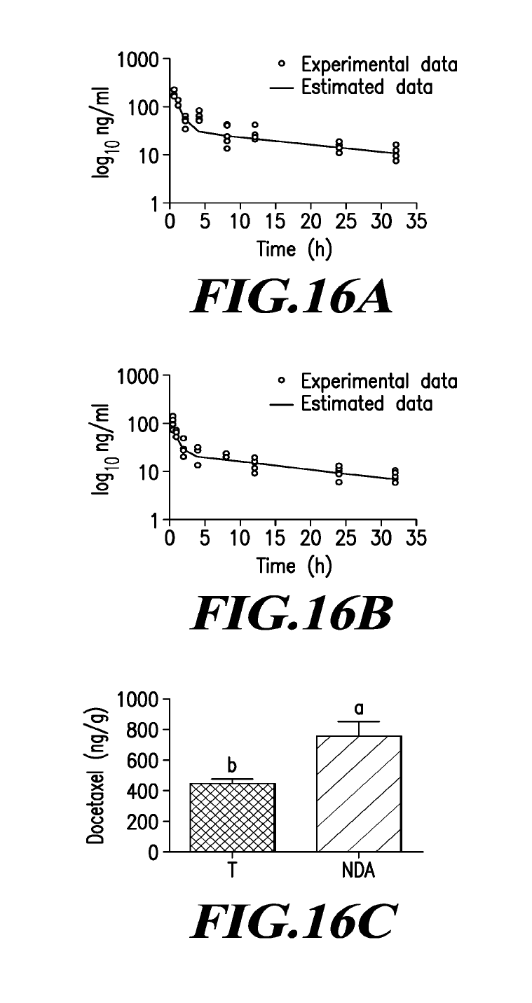

FIG. 16 shows graphs of Docetaxel concentrations in plasma samples at different times (0.5, 1, 2, 4, 8, 12, 24, and 32 hours) following intravenous administration of 2.5 mg/kg DCX to rabbits in the form of TAXOTERE (FIG. 16A) and Nano+DCX+ApoE3 (FIG. 16B).

The primary pharmacokinetic parameters for Docetaxel (TAXOTERE and Nano+DCX+ApoE3) were as follows:

C₁: Coefficient of the first exponential term

k_{d}: Exponent of the first exponential term

C₂: Coefficient of the second exponential term

kₑₗ: Exponent of the second exponential term

For this study, k_{d} and kₑₗ were interpreted as the first order apparent distribution and elimination constants, respectively, and C₁ and C₂ were the coefficients of the adjustment model. The results are provided in Table 9 below.

TABLE 9

| Parameter | Units | TAXOTERE | Nano + DCX + ApoE3 |
|---|---|---|---|
| C₁ | ng/ml | 295.1 | 183.8 |
| k_{d} | h⁻¹ | 1.28 | 1.63 |
| C₂ | ng/ml | 32.5 | 32.2 |
| kₑ₁ | h⁻¹ | 0.035 | 0.037 |

From the estimated values of C₁, k_{d}, C₂, and kₑₗ, the following pharmacokinetic parameters were calculated, as shown in Table 10 below.

t₁/2_{d}: Distribution half-life

t₁/2ₑₗ: Elimination half-life

C_{z}: Coefficient of last exponential term

ABCₒ₋∞: Area under the curve from time zero to infinity

ABC_{tz-}∞: Area under the curve from the last sampling time to infinity

ABCₑₓ: Percentage of area under the curve extrapolated to infinity

TABLE 10

| Parameter | Units | TAXOTERE | Nano + DCX + ApoE3 |
|---|---|---|---|
| t₁/2_{d} | h | 0.54 | 0.42 |
| t₁/2ₑ₁ | h | 19.7 | 18.6 |
| ABC₀₋∞ | h · ng/ml | 1152.0 | 733.6 |
| C_{z} | ng/ml | 10.52 | 7.02 |
| ABC_{tz-}∞ | h · ng/ml | 299.0 | 188.0 |
| ABCₑₓ | % | 26.0 | 25.6 |

Clearance (CI) refers to the volume of blood that has completely cleared of the drug/unit time when it passes through a clearing organ. Results of these measurements are provided in Table 11 below.

TABLE 11

| Formulation | CI = Dose/AUC₀₋∞ (l/h) |
|---|---|
| TAXOTERE | 673 |
| Nano + DCX + ApoE3 | 1056 |

In measuring the liver accumulation, the animals were randomly divided into four groups following the above plasma assay. The groups were as follows:

Group 1 (sample 24 hrs): TAXOTERE formulation, 2.5 mg/kg

Group 2 (sample 24 hrs): Nano+2.5 mg/kg DCX+ApoE3 formulation

Group 3 (sample 36 hrs): TAXOTERE formulation, 2.5 mg/kg

Group 4 (sample 36 hrs): Nano+2.5 mg/kg DCX+ApoE3 formulation

The livers of the rabbits were totally removed at 24 hours and 36 hours, then weighed, frozen and stored at 80° C. The rabbit liver samples were precipitated with acetonitrile, followed by solid phase extraction (SPE), evaporation and resuspension of the resulting extract in a solvent and then analyzed by injection into LC ESI MS/MS. The determinations were performed by liquid chromatography and mass spectrometry (using a Shimadzu UFLC XR liquid chromatograph coupled to a AB Sciex 3200 QTrap mass spectrometer).

FIG. 16C shows concentrations of Docetaxel in rabbits 24 hours after intravenous administration of 2.5 mg/kg) Docetaxel in the form of TAXOTERE and Nano+DCX+ApoE3 (NDA). Values are Media±SEM. (N=5). Values that do not share the same superscript letter are significantly different (p<0.05).

As shown in Table 12 below, the Docetaxel concentrations in liver for NDA were significantly higher than the DCX concentrations after administration of TAXOTERE.

TABLE 12

DCX Concentration in Liver

| Formulation | 24 hours | 36 hours |
|---|---|---|
| TAXOTERE | 446 ± 66 | 495 ± 126 |
| Nano + DCX + ApoE3 | 758 ± 214 | 491 ± 175 |

### Example 7: In Vitro Drug Release of Nanoparticle Formulations

Drug release kinetics were evaluated in vitro by comparing solutions containing:

(a) the nanoparticles with recombinant ApoE3 and loaded with Docetaxel;

(b) the nanoparticles without recombinant ApoE3 and loaded with Docetaxel; and

(c) 0.5 mg/ml TAXOTERE.

The respective solution samples were placed in 12 KDa dialysis bags and dialyzed against a 10% V/V solution of human plasma for 72 hours at 37° C. in an orbital shaker with constant agitation at 60 rpm. Assays were then performed in triplicate, with samples taken from each solution at 0, 1, 2, 3, 6, 9, 24, 48, and 72 hours after dialysis initiation. In each dialyzed phase sample, a Docetaxel extraction was performed using chloroform, and the emulsion resulting from the addition of chloroform was centrifuged to separate the two phases. The recovered organic phase was dried in a rotary evaporator, and the obtained dry material was resuspended in acetonitrile for further HPLC analysis.

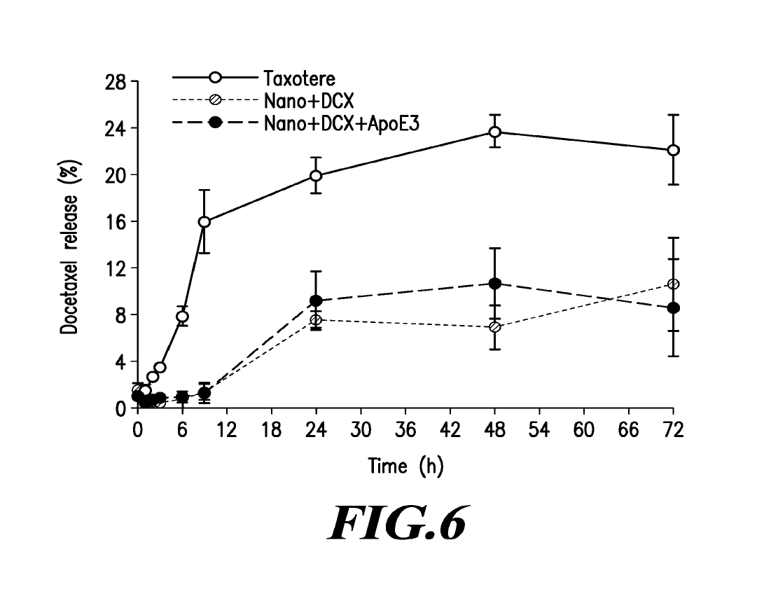

FIG. 6 shows the resulting in vitro Docetaxel release of each solution sample. The values shown are the average±SD.
For solution (a) [nano+DCX+ApoE3], n=4.
For solution (b) [nano+DCX], n=3.
For solution (c) [TAXOTERE], n=6.

The drug-loaded nanoparticles showed sustained drug release for 24 hours with a release percentage of more than 8-10%, thus demonstrating potential suitability as a drug delivery system. The TAXOTERE, on the other hand, released more decetaxel than the lipid nanoparticles. When compared with TAXOTERE, the drug-loaded nanoparticles showed reduced drug release after 72 hours. Additionally, no difference in drug release was shown between solution (a) [containing nanoparticles having a lipid concentration of 20 mg/ml and loaded with 0.2 mg/ml of ApoE3] and solution (b) [containing nanoparticles with the same lipid concentration but not loaded with ApoE3]. As shown by the results, the Docetaxel was retained inside the lipid nanoparticles. Thus, the lipid nanoparticles appear to be able to transport the drug without significant loss.

### Example 8: Tolerability of Lipid Nanoparticles

### Acute Toxicity

To obtain a preliminary characterization for the toxicological properties of lipid nanoparticles excluding known toxicities of Docetaxel, a single-dose tolerability study was performed in healthy New Zealand rabbits and Balb-C mice for the lipid nanoparticles with and without ApoE3, both containing no Docetaxel.

The test animals were maintained under controlled environmental conditions (temperature of 22° C.±1° C.; 12-hour light/dark cycle, light on from 7:00 to 19:00; humidity airflow conditions; and free access to food and water). Acclimatization and quarantine were carried out for minimum period of 10 days prior to the start of the experiment. The animals were permanently identified through the use of caravans.

The experimental design was based on the guidelines: EPA OPPTS 870.1000 Acute Oral Toxicity OECD 423 Acute Oral Toxicity—Acute Toxic Class Methods on which the adaptations to the different routes of administration were made.

New Zealand rabbits received single intravenous doses of lipid nanoparticle (mg/kg animal) of 125 mg/kg; 175 mg/kg; and 200 mg/kg). A control with the same volume of restorative solution was injected in each assay. During the next 10 days, the rabbits were monitored for clinical observation, changes in body weight and blood chemistry.

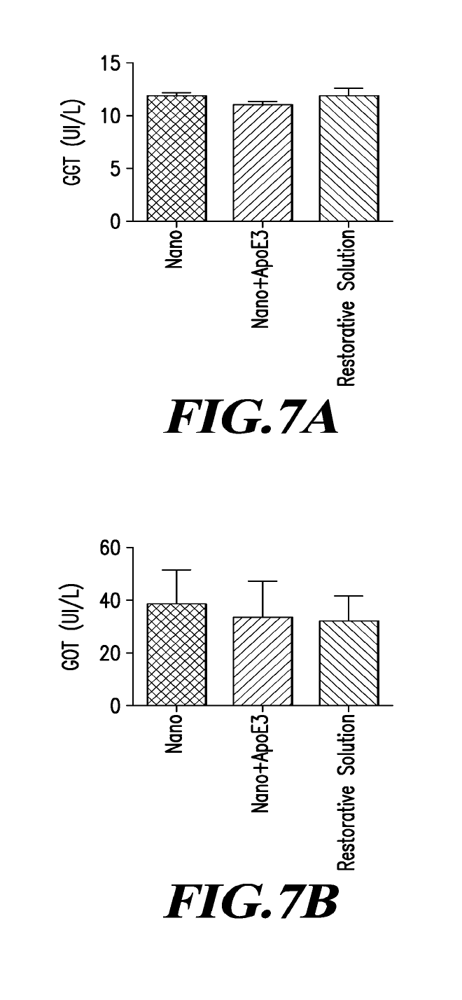

No abnormal clinical observations or appreciable body weight loss were observed. In necropsies performed on animals at the end of the assay, no macroscopic lesions associated with toxicity were observed. Levels of glutamic-oxaloacetic-transaminases (GOT) and gamma-glutamyl-transferase (GGT) were determined after 24 hours for dosages of 125 mg/kg. As shown in FIGS. 7A and 7B, no significant transaminase levels were detected.

Furthermore, the formulations were tested at different dosages. In repeated dose trials, 3 cycles were performed every 7 days with a cumulative dose of lipid nanoparticles of 400 mg of total lipid/kg. Animals presented good general conditions during the 20 days of the trial.

In Balb-C mice, both nanoparticle formulations were tested for a total lipid nanoparticle dosage concentration (mg total lipids/kg animal) of 430 mg/kg; 575 mg/kg; and 715 mg/kg; and a control with the same volume of restorative solution. After administration of nanoparticle formulations, mice were under observation for 11 days. The behavior of the mice was normal throughout the study and no deaths or variances in weight were observed. Transaminases GOT and GGT were determined after 25 hours for 430 mg/kg dose Analogous to the results obtained in the rabbits, mice treated with nanoparticles according to an embodiment of the invention did not exhibit any significant change in transaminase levels with respect to the restorative solution.

In summary, rabbits and mice treated with nanoparticles according to an embodiment of the invention did not exhibit any significant changes in biochemical parameters with respect to the control, suggesting that the developed formulations were well tolerated without any clinical observations suggestive of hypersensitivity or anaphylactic reactions, and not induced hepatotoxicity in rabbits and mice.

### Hepatic Toxicity (GGT and GOT Determination)

The experiment was carried out using New Zealand rabbits kept in facilities under controlled environmental conditions (temperature of 22° C.+3° C.; 12-hour light/dark cycle) and with free access to food and water. Acclimatization was performed for a minimum of 10 days prior to the start of the experiment. Each animal weighted approximately 2.8 kg and was distributed into a group of 5 animals each.

Formulations were administered intravenously (in marginal ear vein) into rabbits that had previously been intravenously injected with a combination of Ketamine-Xylazine-Acepromazine. Amounts of Gamma glutamil transaminase (GGT) and glutamic-oxaloacetic transaminase (GOT) were determined in plasma of the rabbits 24 hours after inoculation with the formulation. The results of each sample were statistically analyzed with ANOVA and Duncan Test using SPSS 11.0.

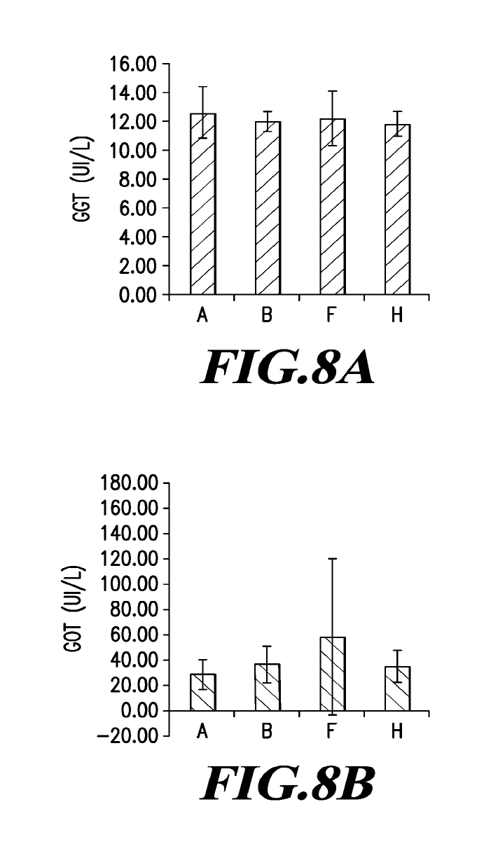

FIG. 8A shows GGT concentration in plasma measured 24 hours after inoculation with (A) Docetaxel (DCX), (B) Nanoparticles (N)+DCX+ApoE, (F) N+DCX, and (H) PBS. The control formulation of DCX was 2.5 mg/kg and the other formulation was used at equivalent concentrations of DCX. No significant differences were observed between the GGT plasma values obtained for the respective formulations.

FIG. 8B shows GOT concentration in plasma measured 24 hours after inoculation with (A) DCX, (B) N+DCX+ApoE, (F) N+DCX, and (H) PBS. The control formulation of DCX was 2.5 mg/kg and the other formulation was used at equivalent concentrations of DCX. O significant differences were observed between the GOT plasma values obtained for the respective formulations. However, a marked variability (SD) was obtained for the results of the N+DCX formulation, which was not observed when the formulation additionally included ApoE (N+DCX+Apo).

### Example 9: Blood Counts and Safety Tests

Tests were performed in New Zealand rabbits to compare the Hemogram profile for: (a) the lipid nanoparticles loaded with Docetaxel, (b) lipid nanoparticles with Docetaxel and with ApoE3; (c) lipid nanoparticle formulation, (d) PBS as a control solution, and (e) TAXOTERE. The intravenous administration was performed in the marginal ear vein and previously rabbits underwent anesthesia intramuscular injection by a combination of Ketamine-Xylazine-Acepromazine (2.4 mg/kg acepromazine, 48 mg/kg Ketamine and 5 mg/kg xylazine)

Blood samples were taken from the central artery of the ear, after sedation. A total of 7 samples were taken for each rabbit, and samples were taken a day before the first doses (−1), and on days 1, 6, 8, 13, 15 and 20 after the first dose. Hemogram results are show in Tables 13 and 14 below.

TABLE 13
Hemogram Analysis Results

| | | | | | | |
|---|---|---|---|---|---|---|
| Sample 1 | Sample 2 | Sample 3 | Sample 4 | Sample 5 | Sample 6 | Sample 7 |

Lipid Nanoparticles Loaded with DCX

| | | | | | | | |
|---|---|---|---|---|---|---|---|
| Hematocrit % | 41 | 39 | 34 | 32 | 27 | 31 | 24 |
| Hemoglobin % | 12 | 11 | 10 | 9 | 8 | 9 | 8 |
| Red Cells × 10⁶/mm³ | 5.8 | 5.5 | 4.8 | 4.6 | 4.2 | 4.3 | 3.8 |
| MCV fL | 70 | 70 | 70 | 69 | 70 | 70 | 65 |
| MCH Pg | 20 | 20 | 20 | 19 | 20 | 20 | 20 |
| MCHC g/dL | 28 | 28 | 29 | 28 | 29 | 28 | 32 |
| White Blood Cells/mm | 7020 | 6400 | 6540 | 5080 | 9140 | 5620 | 6940 |

Lipid Nanoparticles Loaded with DCX and withApoE3
Lipid Nanoparticles Loaded with DCX

| | | | | | | | |
|---|---|---|---|---|---|---|---|
| Hematocrit % | 42 | 41 | 37 | 37 | 24 | 33 | 26 |
| Hemoglobin % | 12 | 12 | 11 | 11 | 8 | 9 | 7 |
| Red Cells × 10⁶/mm³ | 5.8 | 5.6 | 5.1 | 5 | 3.9 | 4.5 | 3.5 |
| MCV fL | 73 | 72 | 72 | 72 | 72 | 72 | 73 |
| MCH Pg | 21 | 21 | 21 | 20 | 20 | 20 | 21 |
| MCHC g/dL | 29 | 29 | 29 | 28 | 29 | 28 | 29 |
| White Blood Cells/mm | 5260 | 5920 | 5640 | 3720 | 6033 | 5500 | 8867 |

Lipid Nanoparticles

| | | | | | | | |
|---|---|---|---|---|---|---|---|
| Hematocrit % | 42 | 40 | 41 | 37 | 35 | 38 | 43 |
| Hemoglobin % | 12 | 11 | 12 | 10.48 | 11 | 11 | 12 |
| Red Cells × 10⁶/mm³ | 5.8 | 5.6 | 5.6 | 5.2 | 5.2 | 5.2 | 5.8 |
| MCV fL | 72 | 72 | 72 | 71.96 | 73 | 73 | 72 |
| MCH Pg | 20 | 20 | 21 | 19.96 | 20 | 20 | 20 |
| MCHC g/dL | 28 | 28 | 28 | 27.88 | 28 | 28 | 28 |
| White Blood Cells/mm | 6400 | 7640 | 6180 | 9700 | 6960 | 7580 | 8020 |

PBS Control Solution

| | | | | | | | |
|---|---|---|---|---|---|---|---|
| Hematocrito % | 40 | 40 | 39 | 40 | 35 | 38 | 42 |
| Hemoglobin % | 12 | 11 | 11 | 11 | 11 | 11 | 12 |
| Red Cells × 10⁶/mm³ | 5.8 | 5.5 | 5.4 | 5.5 | 5.3 | 5.3 | 5.7 |
| MCV fL | 67 | 72 | 72 | 72 | 73 | 72 | 73 |
| MCH Pg | 21 | 20 | 21 | 20 | 21 | 20 | 20 |
| MCHC g/dL | 32 | 28 | 29 | 28 | 29 | 28 | 28 |
| White Blood Cells/mm | 6860 | 7820 | 9100 | 7280 | 6780 | 8360 | 8700 |

The formulations were tested and administered in 4 mg/kg doses (corresponding to 3.2 mg/kg of Docetaxel) and 3 cycles were performed consisting of an injection every 7 days. At day 6 after the first injection, the population of live animals reduced to 50%, with a decrease in the average value of white blood cells from 6720 to 3800.

TABLE 14
Hemogram of Docetaxel Formulation in Rabbits. (Doses of 3.2 mg/kg)

| | Sample 1 | Sample 2 | Sample 3 | Sample 4 | Sample 5 | Sample 6 | Sample 7 |
|---|---|---|---|---|---|---|---|
| Hematocrit % | 40 | 37 | 37 | 39 | 24 | 31 | 19 |
| Hemoglobin % | 11 | 10 | 10 | 11 | 8 | 9 | 8 |
| Red cells × 10⁶/mm³ | 5.7 | 5.2 | 5.2 | 5.6 | 3.9 | 4.4 | 4.2 |
| MCV fL | 71 | 71 | 70 | 69 | 69 | 70 | 45 |
| MCH Pg | 20 | 20 | 20 | 19 | 19 | 20 | 20 |
| MCHC g/dl | 28 | 28 | 28 | 25 | 28 | 29 | 44 |
| White Blood Cells/mm | 6720 | 6175 | 3800 | 3500 | 4000 | 5500 | 2900 |

From these results it can be seen that the active transport mediated by ApoE3 shows a lower toxicity of Docetaxel over the red cells line. This is evidenced when comparing the effect of the treatment with and without ApoE3. For the formulation with Docetaxel without ApoE3 the fall in hematocrit levels and mainly in the VCM during treatment with formulation without ApoE3 is greater than the fall observed in the formulation with ApoE3. After the administration of Docetaxel a fall in all this parameters is expected due to its natural cytotoxicity.

Additional information about the survival of the rabbits was taken after the three cycles of treatment for:
Test Group NA: Nanoparticles with ApoE (4 ml/kg)
Test Group NDA: Nanoparticles with ApoE loaded with 3.2 mg/kg Docetaxel (4 ml/kg)
Test Group T: 3.2 mg/kg Taxotere formulation (4 ml/kg)
   After the first cycle of inoculation, dead animals were found for the T group. After the second cycle of inoculation, dead animals were found for the NDA and T groups. It was further observed that after day 12, animals from both NDA and T groups presented anorexia and adipsia. On the other hand, the NA group showed good general conditions and results during the full 20 day cycle. The results of necropsies performed on the animals found dead during the test are summarized in Table 15 below.

TABLE 15
Signology and Results of Necropsy of Dead Animals

| Group | Signology | Necropsy |
|---|---|---|
| NDA | Diarrhea, | Congestive subcutaneous tissue |
| | anorexia and | Presence of hemorrhagic fluid |
| | adipsia | in the abdominal cavity |
| | | Congestive Kidney and Lungs |
| | | Punctures in liver |
| T | General | Congestive subcutaneous tissue. |
| | Decay. | Presence of hemorrhagic fluid |
| | anorexia and | in the abdominal cavity |
| | adipsia | Congestive lungs |
| | | Punctured liver |
| | | Kidneys with color change |
| | | Congestive bladder |

Based on the above results, Taxotere showed the highest toxicity with a 80% mortality, while the Nanoparticle-ApoE loaded with Docetaxel formulation had a lethality of only 40% at the same time and dosage. No clinical effects were evidenced with the Nanoparticle-ApoE formulation.

### Example 10: 1050 and Selectivity Index

### Immunofluorescence Assay

In order to select cell lines that exhibit more r-LDL on their surface for the “in vitro” test, the following immunofluorescence assay was first performed.

The cell lines were seeded in growth media whereby 50,000 cell/ml were plated in a 96-well plate. After 24 hours, the cells were washed and fixed. A permeabilizing and blocking solution was added prior to incubation with the primary antibody. Cells were then incubated with ab30532 anti-LDL-Receptor, and washed and incubated with a marked antibody (secondary antibodies conjugated to the fluorofor Alexa Fluor 488 ab150081 Goat anti-Rabbit IgG H and L) to give the green color corresponding to the result of the immunofluorescence. For nuclear counterstaining, the cell lines were incubated with 0.05 g/L Hoechst 33342 reagent (Sigma) in PBS solution.

From this assay, the cell lines PC-3, A549 and VERO were selected. PC-3 and VERO were purchased from the American Type Culture Collection (ATCC, Manassas, Va., USA) and A549 from the Asociacion Banco Argentino de Células (ABAC, Buenos Aires, Argentina).

By using in vitro cytotoxicity assays using culture medium with lipoproteins-free serum (SFB), the IC50 (50% inhibitory concentration of the cells) was determined for: (a) TAXOTERE, (b) nanoparticles loaded with Docetaxel, and (c) nanoparticles with ApoE loaded with Docetaxel; for cells PC₃ (prostate cancer epithelial cells A₅₄₉ (lung cancer epithelial cells), and VERO (monkey kidney epithelial cells).

### Cytotoxicity Assays

50 μL of the cell suspensions were seeded in 96-well plates in duplicate with Dulbecco's basal medium plus 10% fetal bovine serum supplemented with 10% fetal bovine serum (FBS) for PC-3, and with DMEM/Ham's F12 medium containing 10% FBS, 1% 1-glutamine for A549 cell line. Both were maintained in culture for 24 hours. The cells were then treated with serial diluted concentrations of DCX starting from 100 nM and incubated for 48 hours. At the end of incubation, 20 μL/well of MTS/PMs solution (2 mL of MTS with 100 μL of PMS solution) were added. Then, plates were incubated at 37° C., 5% CO₂ for 4 hours. The plates were revealed with a microplate reader at 492 nm and 690 nm, respectively. The result allowed for conditions for evaluating the growth inhibition of the Nano+DCX and Nano+DCX+ApoE3 to be defined.

TABLE 16
IC50 in Cells Cultured With Serum Free of Lipoprotein

| | | | | | | |
|---|---|---|---|---|---|---|
| Test 1 | Test 2 | Test 3 | Test 4 | Test 5 | Test 6 | Test 7 |

VERO CELLS

| | | | | | | | | IC50 in VERO |
|---|---|---|---|---|---|---|---|---|
| Sample | 170703 | 170705 | 170710 | 170711A | 170711B | 170717A | 170717B | (nM) |
| DCX | 137.5 | 106 | 105.5 | 140 | 153 | 152.5 | 111 | 129.4 ± 8.06 |
| NANO + DCX | 124 | 162 | 123.5 | 270 | 258.5 | 178 | 155.5 | 181.64 ± 22.6 |
| NANO + DCX + | | 197 | 243.5 | 236.5 | 258.5 | 250 | 207.5 | 199 ± 34.2 |
| APOE | | | | | | | | |

PC3 CELLS

| | | | | | IC50 in PC3 |
|---|---|---|---|---|---|
| Sample | 170703 | 170705 | 170717A | 170807 | (nM) |
| DCX | 37.5 | 34.5 | 39 | 10 | 30.25 ± 6.3 |
| NANO + DCX | 29 | 43.5 | 15 | 11 | 24.6 ± 7.4 |
| NANO + DCX + | 27.5 | 20 | 16.5 | | 20 ± 7.8 |
| APOE | | | | | |

A549 CELLS

| | | | | | | | IC50 in A549 |
|---|---|---|---|---|---|---|---|
| Sample | 170710 | 170711B | 170717A | 170717B | 170720A | 170720B | (nM) |
| DCX | 25.5 | 33 | 37.5 | 45.5 | 36.5 | 25 | 33.8 ± 3.2 |
| NANO + DCX | 31 | 22.5 | 37.5 | 40 | 19.5 | 22 | 28.8 ± 3.6 |
| NANO + DCX + | 19 | 18.5 | 14 | 15.5 | 14 | 14.5 | 15.91 ± 0.93 |
| APOE | | | | | | | |

TABLE 17
IC50 in Cells Cultured With Normal Serum

| | | | | | | | |
|---|---|---|---|---|---|---|---|
| | | | | | | | IC50 in VERO |
| | | | | | | | Average |
| Test 1 | Test 2 | Test 3 | Test 4 | Test 5 | Test 6 | Test 7 | (nM) |

VERO CELLS

| | | | | | IC50 in VERO |
|---|---|---|---|---|---|
| Sample | 170707 | 170710 | 170711 | 170711 | (nM) |
| DCX | 136.5 | 145 | 139.5 | 149.5 | 142.6 ± 2.9 |
| NANO + DCX | 155.5 | 264.5 | 247.5 | 190 | 214.9 ± 25.3 |
| NANO + DCX + | | 402 | 384.5 | 620.5 | 337 ± 35.7 |
| APOE | | | | | |

PC3 CELLS

| | | | | | IC50 in PC3 |
|---|---|---|---|---|---|
| Sample | 170418 | 170420 | 170421 | 170424 | (nM) |
| DCX | 3.5 | 3.9 | 3.3 | 3.4 | 3.5 ± 0.1 |
| NANO + DCX | 3.85 | 6.8 | 4.15 | 6.25 | 5.3 ± 0.7 |
| NANO + DCX + | 4.75 | 8 | 6.15 | 9.5 | 7.9 ± 0.8 |
| APOE | | | | | |

A549 CELLS

| | | | | | IC50 in A549 |
|---|---|---|---|---|---|
| Sample | 140418 | 140420 | 140421 | 140424 | (nM) |
| DCX | 8.75 | 6.65 | 9.6 | 9.9 | 8.7 ± 0.7 |
| NANO + DCX | 13.3 | 11 | 13.25 | 12.2 | 12.5 ± 0.5 |
| NANO + DCX + | 18.2 | 21.7 | 17.25 | 18 | 19 ±1.2 |
| APOE | | | | | |

Since it is not conventionally correct to compare IC50 results, an index of selectivity was instead calculated based on the obtained results. Based on the above IC50 results, the SI was calculated in order to determine the cytotoxic selectivity of the substances tested pursuant to the following equation:

SI=IC50 (in non-cancer cells)/IC50 (in cancer cells)

The SI shows the differential activity of a compound; the higher the SI value, the more selective it will be.

TABLE 18
Selectivity Index (SI)

| | NORMAL SERUM | | | | | LP-FREE SERUM | | | | |
|---|---|---|---|---|---|---|---|---|---|---|
| | IC50 | | | | | IC50 | | | | |
| | Normal | | | | | Normal | | | | |
| | Cell | IC50 | IC50 | SI | SI | Cell | IC50 | IC50 | Si | Si |
| | (Vero) | A549 | PC3 | A549 | PC3 | (Vero) | A549 | PC3 | A549 | PC3 |
| DCX | 143 | 9 | 4 | 16 | 41 | 129 | 34 | 30 | 4 | 4 |
| NANO + DCX | 214 | 12 | 7 | 17 | 31 | 182 | 33 | 28 | 6 | 6 |
| NANO + DCX + | 337 | 19 | 7 | 18 | 47 | 232 | 16 | 21 | 15 | 11 |
| APOE | | | | | | | | | | |

The SI value found for NANO+DCX+APOE in cancer cell (A549 and PC3) tests with lipoprotein-free serum is >10. Thus, according to the selectivity criterion, it could be said that this formulation would be selective for these cells. For the tests performed with cells cultured in normal serum, both the DCX and NANO+DCX+APOE formulations were found to have values higher than 10; however, the NANO+DCX+APOE formulation showed slightly higher SI values than the DCX.

Growth Inhibition by DCX, Nano+DCX, and Nano+DCX+ApoE3

Absorbance values obtained from the in vitro cytotoxicity assay of DCX, Nano+DCX, and Nano+DCX+ApoE3 samples were plotted based on the DCX concentration employed, using OriginPro 8 software (ORIGINLAB Corporation, USA). The IC50 (nM) of each of the samples was determined at 50% of the maximum absorbance value obtained, corresponding to the value obtained for the growth control. The anti-proliferation effect shown on different cells with DCX, Nano+DCX, and Nano+DCX+ApoE3 decreased cell proliferation.

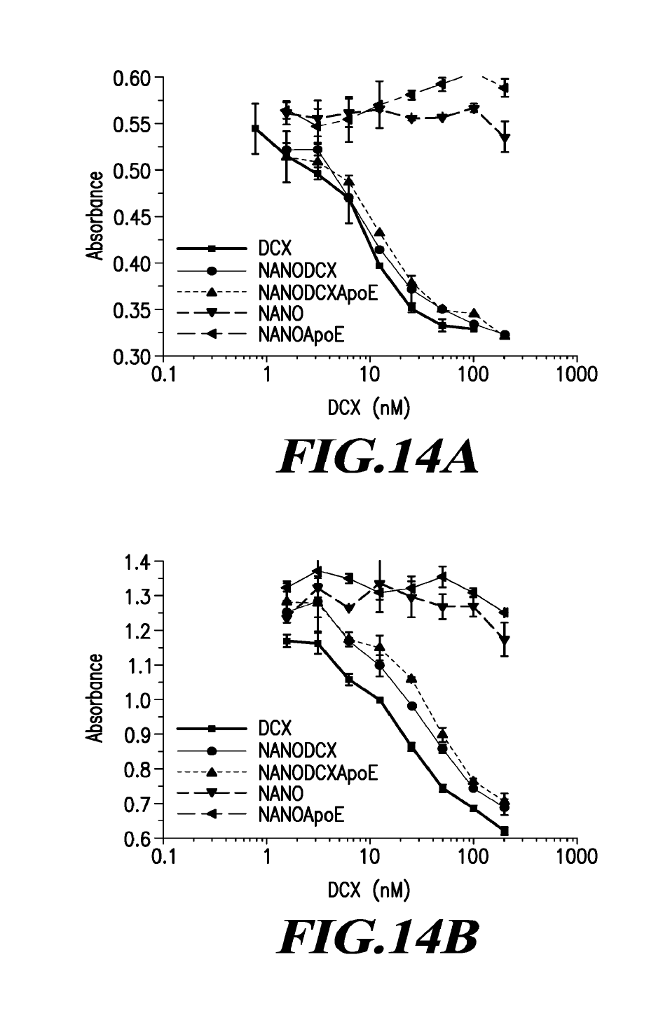

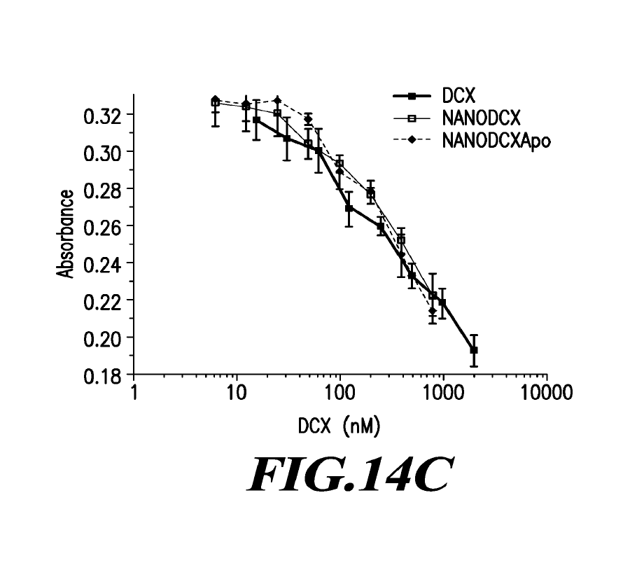

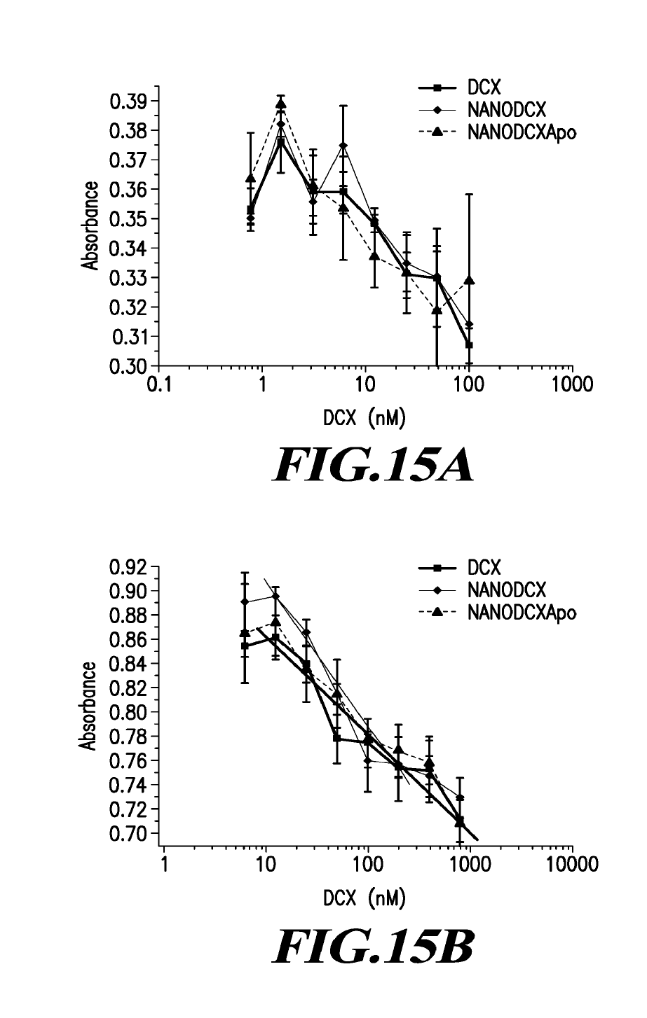

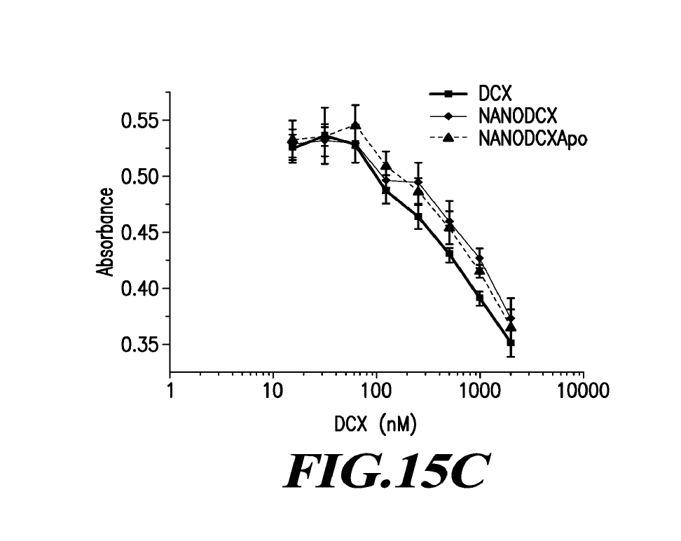

The absorbance values based on the DCX concentration employed are plotted in FIGS. 14A-C. FIGS. 15A-C show additional graphs of absorption versus DCX concentration of Experiment 170418-AG for (a) PC-3 cells and (b) A549 cells and Experiment 170705 for (c) VEERO cells. The experiments were repeated four times in order to obtain independent IC50 values for each of the samples. The results are summarized in table 18 above.

### Example 11: Inmunogenicity in Mice

ELISA was used to study anti Apo Indirect antibodies in different species. A test of immunogenicity in mice was mapped out and implemented.

For the ELISA assay, seven groups of 5 mice of Balb/c strain provided by the Centro de Medicina Comparada of the UNL of 6 weeks old were used.

At day 0, a pre-immune serum sample was obtained from each animal by puncture of the maxillary sinus under sedation. Each group of animals was inoculated with decreasing amounts of apoprotein ApoE3 resuspended in sterile water for injection according to the following:
Group 1 (it is the positive control group): 1 mg of ApoE3 per kg of animal plus adjuvant of Freund (complete form: first inoculation or incomplete form: second to fourth inoculation).
Group 2: 1 mg of ApoE3 per kg of animal without adjuvant.
Group 3: 0.5 mg of ApoE3 per kg of animal without adjuvant.
Group 4: 0.25 mg of ApoE3 per kg of animal without adjuvant.
Group 5: 0.125 mg of ApoE3 per kg of animal without adjuvant.
Group 6: 0.0625 mg of ApoE3 per kg of animal without adjuvant.
Group 7: 0.03125 mg of ApoE3 per kg of animal without adjuvant.
   Subcutaneous inoculations of a volume between 0.1 and 0.2 ml per animal were made at 2 to 4 sites of administration. This immunization scheme was repeated on days 21, 42 and 63 of the protocol. Fourteen days after of the 2nd immunization (day 35) a blood sample sinus puncture was obtained, proceeding in the same manner on day 56. The animals were completely bled by cardiac puncture on day 77 of the immunization protocol, following the procedures in accordance with the provisions of the Guidelines for the care and use of animals. The described protocol is detailed in the timeline provided in Table 20 below.

The results of the optical density of the indirect ELISA tests on the serum from different animals of the 7 groups after the fourth inoculation are graphed in FIGS. 16A-16F.

As depicted in FIG. 16, the ELISA results show that the ApoE3 use in the formulation does not trigger a specific antibody mediated immune. The OD levels observed for the tests groups are much lower than the results observed for the positive control Group. Furthermore, even for 100 fold higher serum concentrations, the OD levels obtained by the hyper-immune serum (positive control) are not reached. T immunogenicity of this human ApoE would be expected to be very low in other species.

### REFERENCES

Each of the references listed below and referenced in the disclosure is incorporated herein by reference in its entirety.
Wagner et al., “Uptake Mechanism of ApoE-Modified Nanoparticles on Brain Capillary Endothelial Cells as a Blood-Brain Barrier Model,” PLoS ONE 7(3): e32568 (Mar. 1, 2012).
Kreuter et al., “Apolipoprotein-Mediated Transport of nanoparticle-Bound Drugs Across the Blood-Brain Barrier,” Journal of Drug Targeting, 10(4): 317-325 (2002).
Dehouck, Bénédicte, et al. “A new function for the LDL receptor: transcytosis of LDL across the blood-brain barrier.” The Journal of cell biology 138.4: 877-889 (1997).
Versluis, A. Jenny, et al. “Stable incorporation of a lipophilic daunorubicin prodrug into apolipoprotein E-exposing liposomes induces uptake of prodrug via low-density lipoprotein receptor in vivo.” Journal of Pharmacology and Experimental Therapeutics 289.1: 1-7 (1999).
Liu, Chia-Chan, et al. “Apolipoprotein E and Alzheimer disease: risk, mechanisms and therapy.” Nature Reviews Neurology 9.2: 106-118 (2013).
Vila-Rodriguez, Fidel, and William G. Honer. “ApoE and cholesterol in schizophrenia and bipolar disorder: comparison of grey and white matter and relation with APOE genotype.” Journal of psychiatry & neuroscience: JPN 36.1:47 (2011).
BETTS et al. Environmental Toxicology and Chemistry, Vol. 32, No. 4, pp. 889-893
Narvekar, Mayuri, et al. “Nanocarrier for poorly water-soluble anticancer drugs—barriers of translation and solutions.” AAPS Pharm. SciTech 15.4: 822-833 (2014).
Gad, Shayne Cox, ed. Pharmaceutical manufacturing handbook: production and processes. Vol. 5. John Wiley & Sons, 2008.
Lockman, P. R., et al. “Nanoparticle technology for drug delivery across the blood-brain barrier.” Drug development and industrial pharmacy 28.1 (2002): 1-13.
Mims, Martha P., Maurizio R. Soma, and Joel D. Morrisett. “Effect of particle size and temperature on the conformation and physiological behavior of apolipoprotein E bound to model lipoprotein particles.” Biochemistry 29.28: 6639-6647 (1990).
Hatters, Danny M., Clare A. Peters-Libeu, and Karl H. Weisgraber. “Apolipoprotein E structure: insights into function.” Trends in biochemical sciences 31.8: 445-454 (2006).
Peters-Libeu, Clare A., et al. “Model of biologically active apolipoprotein E bound to dipalmitoylphosphatidylcholine,” Journal of Biological Chemistry 281.2 (2006): 1073-1079.
Robitaille, N., et al. “Apolipoprotein E polymorphism in a French Canadian population of northeastern Quebec: allele frequencies and effects on blood lipid and lipoprotein levels.” Human biology, pp. 357-370 (1996).
Valdez, Rodolfo, et al. “Apolipoprotein E polymorphism and insulin levels in a biethnic population.” Diabetes Care 18.7: 992-1000 (1995).
Haffner, Steven M., et al. “Apolipoprotein E polymorphism and LDL size in a biethnic population.” Arteriosclerosis, thrombosis, and vascular biology 16.9: 1184-1188 (1996).
Utermann, Gerd. “Apolipoprotein E polymorphism in health and disease.” American heart journal 113.2: 433-440 (1987).
Feng, Lan, et al. “Development and optimization of oil-filled lipid nanoparticles containing docetaxel conjugates designed to control the drug release rate in vitro and in vivo.” Int. J. Nanomedicine 6: 2545 (2011).
Lundberg, B. B., et al. “A lipophilic paclitaxel derivative incorporated in a lipid emulsion for parenteral administration.” Journal of Controlled Release 86.1: 93-100 (2003).
Rodrigues, Debora G. et al., “Improvement of paclitaxel therapeutic index by derivatization and association to a cholesterol-rich microemulsion: in vitro and in vivo studies.” Cancer chemotherapy and pharmacology 55.6: 565-76 (2005).
Domb, Abraham J., Joseph Kost, and David Wiseman, eds. Handbook of biodegradable polymers. Vol. 7. CRC Press, 1998.
Patel, Hitesh R., Rakesh P. Patel, and M. M. Patel. “Poloxamers: A pharmaceutical excipients with therapeutic behaviors.” International Journal of PharmTech Research 1.2: 299-303 (2009).
Frim, David M., et al. “The surfactant poloxamer-188 protects against glutamate toxicity in the rat brain.” Neuroreport 15.1: 171-74 (2004).
Coors, Esther A., et al. “Polysorbate 80 in medical products and nonimmunologicanaphylactoid reactions.” Annals of Allergy, Asthma & Immunology 95.6: 593-99 (2005).
Chaturvedi, S. P., and Vimal Kumar. “Production techniques of lipid nanoparticles: a review.” RJPBCS 3.3: 525-541 (2012).
Lasa-Saracibar, Beatriz, et al. “Lipid nanoparticles for cancer therapy: state of the art and future prospects.” Expert opinion on drug delivery 9.10: 1245-1261 (2012).
Hu, Fu-Qiang, et al. “Preparation and characteristics of monostearin nanostructured lipid carriers.” International Journal of Pharmaceutics 314.1: 83-89 (2006).

1. A lipid nanoparticle, comprising a combination of:
a phospholipid component,
a triglyceride component,
a cholesterol ester component,
a free cholesterol component,
an apolipoprotein component, and
a therapeutic agent,
wherein:
the components are distributed so as to form a lipid core, surrounded by a phospholipid layer and a surfactant coating layer;
the apolipoprotein component is human recombinant E3 apolipoprotein (ApoE3) and is bonded to a surface of the nanoparticle without Polysorbate 80; and
the nanoparticle has an average size between 20 and 150 nm, a Z-potential between −25 and −5 my, and a PDI dispersion value between 0.08 and 0.30.

2. (canceled)

3. (canceled)

4. The lipid nanoparticle according to claim 1, wherein the lipid core is formed of the cholesterol ester and triglyceride components, and the triglyceride component is selected from the group consisting of castor oil, coconut oil, and soybean oil.

5. (canceled)

6. (canceled)

7. The lipid nanoparticle according to claim 4, wherein the lipid core is formed of cholesterol ester and castor oil, and the therapeutic agent is dissolved therein.

8. The lipid nanoparticle according to claim 1, wherein the therapeutic agent is an anticancer drug.

9. The lipid nanoparticle according to claim 8, wherein the anticancer drug is selected from the group consisting of azacitidine, bendamustine, carmustine, cabacitaxel, cisplatin, cytarabine, docetaxel, doxonibicine, eribulin, estramustine, etoposide, floxuridine, gemcitabine, melphalan, metotrexate, paclitaxel, oxaliplatin, romidepsin, SN-38, vincristine and vinorelvine.

10. The lipid nanoparticle according to claim 9, wherein the therapeutic agent is Docetaxel, and the nanoparticle, in a culture with lipoprotein-free serum, has a lower IC50 and a higher selectivity index in cancer cells as compared to Docetaxel in its original formulation with Polysorbate 80.

11. The lipid nanoparticle according to claim 1, wherein the nanoparticle, in a culture with lipoprotein-free serum, has a lower IC50 and a higher selectivity index in cancer cells, as compared to non-cancer cells.

12. The lipid nanoparticle according to claim 10, wherein a mass ratio of Docetaxel to ApoE3 in the nanoparticle is from 1.1 to 3.3 of Docetaxel to ApoE3.

13. The lipid nanoparticle according to claim 10, wherein a molar ratio of Docetaxel molecules per each recombinant ApoE3 molecule in the nanoparticle is from 45 to 140.

14. (canceled)

15. The lipid nanoparticle according to claim 1, wherein the phospholipid and triglyceride components are present in the nanoparticle in a weight ratio ranging from [5.25-8.27] to [3.75-12.1] by weight of the nanoparticle.

16. (canceled)

17. The lipid nanoparticle according to claim 15, wherein the weight ratio of the phospholipid to triglyceride components provides a therapeutic agent encapsulation efficiency of the nanoparticle of over 90%, as determined by HPLC.

18. The lipid nanoparticle according to claim 1, wherein the phospholipid, triglyceride, cholesterol ester, cholesterol, ApoE3, and therapeutic agent components are respectively present in the nanoparticle in a weight ratio ranging from [5.25-8.27]:[3.75-12.1]:[0-0.6]:[0-0.9]:[0.1-1.4]:[0.3-0.9], respectively.

19. A pharmaceutical composition, comprising:
a therapeutically effective amount of the lipid nanoparticles according to claim 1; and
a pharmaceutically acceptable excipient.

20. The pharmaceutical composition according to claim 19, wherein the pharmaceutically acceptable excipient is selected from the group consisting of sucrose, sodium taurodeoxycholate, Poloxamer 188, sodium acyl phosphate, potassium dihydrogen phosphate, sodium chloride and potassium chloride.

21. The pharmaceutical composition according to claim 19, wherein the therapeutic agent is Docetaxel and the composition does not contain Polysorbate 80.

22. The pharmaceutical composition according to claim 19, wherein the composition is a lyophilized composition that is stable for at least 18 months at 25° C.

23. (canceled)

24. A pharmaceutical composition that has been reconstituted from the lyophilized composition according to claim 22, therein the therapeutic agent is Docetaxel and the composition does not contain Polysorbate 80, and wherein a molar ratio of Docetaxel molecules per each recombinant ApoE3 molecule in the reconstituted composition is from 45-140.

25. A kit comprising:
the lyophilized composition according to claim 22; and
a sterile aqueous solution for mixing the lyophilized composition prior to use.

26. A method of cancer therapy, comprising:
treating a cancer tissue with the nanoparticles according to claim 1, wherein the nanoparticles are loaded with and deliver an effective dosage of Docetaxel as the therapeutic agent to the cancer tissue via r-LDL-mediated endocytosis.

27. A method of treating cancer in a patient, comprising:
administering to the patient the pharmaceutical composition according to claim 19.

28. (canceled)

29. The method according to claim 27, wherein the cancer is lung cancer or prostate cancer, and the lipid nanoparticles are loaded with Docetaxel as the therapeutic agent without Polysorbate 80.

30. (canceled)

30. A method of manufacturing the lipid nanoparticles according to claim 1, comprising:
preparing an organic phase comprising the phospholipid, triglyceride, cholesterol ester, and free cholesterol components and the therapeutic agent;
preparing an aqueous phase comprising water, Poloxamer 188, sodium taurodeoxicolate, sodium taurocolate and choline chloride;
injecting the organic phase into the aqueous phase through an injection nozzle in a highly turbulent regime to obtain the lipid nanoparticles;
concentrating the lipid nanoparticles to the appropriate concentration of total lipids; and
adding ApoE3 in aqueous solution to the obtained nanoparticles.

31. (canceled)

32. The method of manufacturing the lipid nanoparticles according to claim 30, wherein the method is a low energy process due to rapid diffusion of surfactant and/or solvent molecules from the organic phase of the continuous phase during nano-emulsion formation.

33. A composition, comprising:
lipid nanoparticles in lipoprotein free serum, the lipid nanoparticles being coated with human recombinant E3 lipoprotein (ApoE3), and loaded with Docetaxel without any Polysorbate 80,
wherein the composition has a lower IC50 inhibitory concentration and a higher selectivity index than Docetaxel in its regular formulation with Polysorbate 80.
